(19) United States
(12) Patent Application Publication
(10) Pub. No.: US 2021/0025894 A1
(43) Pub. Date: Jan. 28, 2021

(54) DIAGNOSTIC ASSAYS TO DETECT TUMOR ANTIGENS IN CANCER PATIENTS

(71) Applicant: Hoffmann-La Roche Inc., Little Falls, NJ (US)

(72) Inventors: Diana DAROWSKI, Schlieren (CH); Camille Loise Sophie DELON, Mulhouse (FR); Lydia Jasmin HANISCH, Birmensdorf (CH); Christian KLEIN, Bonstetten (CH); Christian JOST, Zurich (CH); Ekkehard MOESSNER, Kreuzlingen (CH); Vesna PULKO, Zurich (CH); Wei XU, Schlieren (CH)

(73) Assignee: Hoffmann-La Roche Inc., Little Falls, NJ (US)

(21) Appl. No.: 17/062,270

(22) Filed: Oct. 2, 2020

Related U.S. Application Data
(63) Continuation of application No. PCT/EP2019/058214, filed on Apr. 2, 2019.

(30) Foreign Application Priority Data
Apr. 4, 2018 (EP) 18165601.8

(51) Int. Cl. G01N 33/574; G01N 33/50

(52) U.S. Cl. CPC G01N 33/57484; G01N 33/505

(57) ABSTRACT

The present invention generally relates to diagnostic assays using cell cultures, in particular to chimeric antigen receptor (CAR) expressing reporter cell assays to analyze samples, in particular patient samples, to diagnose cancer by quantifying the expression of tumor antigens and/or predicting clinical response to cancer immunotherapies. A further aspect of the present invention is to improve safety of e.g., cancer immunotherapies.

### CROSS REFERENCE TO RELATED APPLICATIONS

This application is a continuation of International Application No. PCT/EP2019/058214, filed Apr. 2, 2019, which claims benefit to European Patent Application No. 18165601.8, filed Apr. 4, 2018, all of which are hereby expressly incorporated by reference in their entirety as though fully set forth herein.

### SEQUENCE LISTING

This application contains a Sequence Listing which has been submitted electronically in ASCII format and is hereby incorporated by reference in its entirety. Said ASCII copy, created on Sep. 23, 2020, is named P34754_US_Sequence_listing and is 75,296 bytes in size.

### FIELD OF THE INVENTION

The present invention generally relates to diagnostic assays using cell cultures, in particular to chimeric antigen receptor (CAR) expressing reporter cell assays to analyze samples, in particular patient samples, to diagnose cancer by quantifying the expression of tumor antigens and/or predicting clinical response to cancer immunotherapies. A further aspect of the present invention is to improve safety of e.g., cancer immunotherapies.

### BACKGROUND

Cancer is one of the leading causes for death throughout all age cohorts. Cancer is an abnormal stage of cells which leads to uncontrolled proliferation of one or more cell populations. Ultimately, the proliferation leads to aberration of normal biological function leading to a plurality of clinical and non-clinical symptoms. Tumor cells typically display one or several of properties which distinguish the tumor cells from normal cells, such as morphology, expression of fetal antigens, lack of contact inhibition and growth-factor independence.

Unfortunately, most cancers are asymptomatic at an early stage of the disease leading to the challenging situation that, e.g., for lung cancers, only 15% are found at an early, still localized stage. However, for such early diagnosed patents, the five-year survival rate can be as high as 85% whereas only 2% of such patients survive 5 years after the cancer has spread to other organs. Despite of decades of encouraging improvements to cancer therapy, the most significant predictor for successful treatment of cancer remains early detection and classification of the disease.

Typically, the immune system does not alarm and trigger recognition of tumor cells owing to the nature of these cells originated from the “self”. Cancer immunotherapies are aimed to harness the immune system to target tumor cells by recognizing unique proteins exclusively expressed by tumors, and simultaneously engaging immune cell action e.g. via antibody-dependent cytotoxicity (ADCC) or T cell cytotoxicity which enables destruction of the tumor cells. This engagement can be achieved via classical ADCC-competent and/or T cell bispecific antibodies, or T cells engineered to express the native T-cell receptor (TCR-T) recognizing the tumor antigen or an artificial chimeric antigen (CAR-T). The antibodies recognize either conventional tumor surface proteins or protein-derived peptides presented in the context of MHC complex (pMHC). In either approach, not all the patients respond to the therapies in the clinics as the density of the tumor antigens expressed on the tumor cell surface or pMHC varies largely among patients across all cancer types.

Therefore, a valid tool to detect and quantify the amount of tumor antigens (surface and/or pMHC) would warrant a better diagnosis of cancer patients, and could predict the likelihood of the clinical response to the respective cancer immunotherapies. Furthermore, cancer immunotherapies can sometimes trigger unwanted immune response targeting normal tissues. Early prediction of the safety of immunotherapies would be helpful for the physicians to monitor the potential lethal side effect in patients.

The detection of tumor antigens in diverse body fluids remains of utmost importance given, e.g., that the presence of already a very small number of viable tumor cells in lymph or blood may indicate an important hall mark in the disease progression. Immunological diagnostic assays provide an important tool towards this end capable of detecting a variety of disease conditions. However, such assays may not always be sensitive and/or specific enough to reliably detect tumor cells, e.g., in the context of MHC-presented protein-derived peptides.

Accordingly, there remains a need for highly sensitive and robust diagnostic assays to detect cancer antigens suitable for the use in detecting malignant cells and/or to predict on-target off-tissue effects to improve safety of immunotherapies and to predict response of a patent to a particular immunotherapeutic.

The present inventors developed a highly flexible assay with an integrative and straight-forward readout feasible for high-throughput formats to screen tumor antigens in cancer patients applicable to both classical surface cancer antigens and MHC complex presented protein-derived peptides. This invention applies to diagnosis of cancer patients, prediction of the clinical response to the immunotherapies, and safety measurement of the immunotherapies.

### SUMMARY OF THE INVENTION

The present invention generally relates to diagnostic assays for determining the presence of a target antigen, e.g., a tumor target antigen and/or a tumor cell in a sample, particularly in a sample derived from a patient, and combines the detection of target antigen with the activation of reporter cells in response to tumor cells. The assays of the present invention are suitable to screen patient samples and allow precise measurement of antigen density in the context of surface antigens and/or MHC presented protein-derived peptides. The assays of the present invention are further useful for predicting the likelihood of the clinical response to cancer immunotherapies.

Accordingly, herein provided is a diagnostic assay for determining the presence of a tumor cell in a sample, the diagnostic assay comprising the steps of:
a) contacting the sample with a chimeric antigen receptor (CAR) expressing reporter T (CAR-T) cell wherein the reporter CAR-T cell comprises:
   i. a CAR capable of specific interaction with the tumor cell, wherein the CAR is operationally coupled to a response element;
   ii. a reporter gene under the control of the response element; and
b) determining T cell activation by measuring the expression of the reporter gene to establish the presence of the tumor cell.

In one embodiment, the CAR comprises a target antigen binding moiety capable of specific binding to a tumor target antigen.

In one embodiment, the target antigen binding moiety is a Fab fragment.

In one embodiment, the reporter T cell is a Jurkat cell.

In one embodiment, the tumor target antigen is a cell surface antigen and/or receptor.

In one embodiment, the tumor target antigen is selected from the group consisting of CD20, CD38, CD138, CEA, EGFR, Fo1R1, HER2, LeY, MCSP, STEAP1, TYRP1, and WT1, or a fragment thereof.

In one embodiment, the tumor target antigen is a peptide bound to a molecule of the human major histocompatibility complex (MHC).

In one embodiment, the target antigen binding moiety is a T cell receptor like (TCRL) antigen binding moiety.

In one embodiment, the CAR comprises at least one intracellular stimulatory signaling and/or co-stimulatory signaling domain.

In one embodiment, activation of the intracellular signaling and/or co-signaling domain leads to activation of the response element.

In one embodiment, activation of the response element leads to expression of the reporter gene.

In one embodiment, the response element is part of the NFAT pathway, the NF-κB pathway or the AP-1 pathway.

In one embodiment, the reporter gene is coding for a luminescent protein.

In one embodiment, the reporter gene is coding for green fluorescent protein (GFP) or luciferase.

In one embodiment, the sample is a patient sample derived from an individual suffering from a disease, in particular wherein the disease is cancer.

In one embodiment, provided is a method for predicting the efficacy of an antitumor CAR-T cell treatment, comprising providing a sample from a subject having a tumor, and determining T cell activation by measuring the expression of the reporter gene according to the diagnostic assay of any one of embodiments 1 to 15, wherein activation of the reporter gene is indicative for efficacy of the antitumor CAR-T cell treatment when applied to the subject.

### SHORT DESCRIPTION OF THE FIGURES

FIG. 1 depicts a schematic representation of the diagnostic Jurkat NFAT reporter CAR-T cell assays. A tumor associated antigen (TAA) can be recognized by the anti-TAA antigen binding receptor expressing Jurkat NFAT reporter CAR-T cell. This recognition leads to the activation of the cell which can be detected by measuring luminescence.

FIG. 2A to 2F depicts the architecture of different CAR formats used in the present invention. FIG. 2A shows the architecture of the Fab format. Depicted is the extracellular domain comprising an antigen binding moiety which consists of an Ig heavy chain fragment and an Ig light chain. Attached to the heavy chain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 2B shows the architecture of the Fab format with heavy and light chain swap. Depicted is the extracellular domain comprising an antigen binding moiety which consists of an Ig heavy chain fragment and an Ig light chain. Attached to the light chain constant domain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 2C shows the architecture of the scFab format. Depicted is the extracellular domain comprising an antigen binding moiety which consists of an Ig heavy chain fragment and an Ig light chain, both connected by a linker. Attached to the heavy chain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 2D shows the architecture of the crossFab format with VH-VL swap. Depicted is the extracellular domain comprising an antigen binding moiety which consists of an Ig heavy chain fragment and an Ig light chain wherein the VH and VL domains are exchanged. Attached to the heavy chain constant domain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 2E shows the architecture of the crossFab format with CH-CL swap. Depicted is the extracellular domain comprising an antigen binding moiety which consists of an Ig heavy chain fragment and an Ig light chain wherein the CH and CL domains are exchanged. Attached to the light chain constant domain, a linker connects the antigen recognition domain with an anchoring transmembrane domain (ATD) which is fused to an intracellular co-stimulatory signaling domain (CSD) which in turn is fused to a stimulatory signaling domain (SSD). FIG. 2F shows the architecture of the classic scFv format with an extracellular antigen recognition domain, consisting of a variable heavy and variable light chain, both connected by a linker. Attached to the variable light chain, a linker connects the antigen recognition domain with an anchoring transmembrane domain (ATD) which is fused to an intracellular co-stimulatory signaling domain (CSD) which in turn is fused to a stimulatory signaling domain (SSD).

FIG. 3A to 3F depicts a schematic representation illustrating the modular composition of exemplary expression constructs encoding CARs used according to the invention. FIG. 3A and FIG. 3B depict exemplary Fab formats. FIG. 3C depicts an exemplary scFab format. FIG. 3D and FIG. 3E depict exemplary crossFab formats. FIG. 3F depicts a classic scFv format.

FIG. 4 depicts a diagnostic Jurkat NFAT reporter CAR-T cell assay using CD20 expressing SUDHDL4 tumor cells as target cells. A single clone of anti-CD20-Fab-CD28ATD-CD28CSD-CD3zSSD expressing Jurkat NFAT reporter CAR-T cells was used as reporter cells.

FIG. 5 depicts a diagnostic Jurkat NFAT reporter CAR-T cell assay using CD20 expressing SUDHDL4 tumor cells as target cells. A pool of anti-CD20-Fab-CD28ATD-CD28CSD-CD3zSSD or anti-CD20-crossFab-CD28ATD-CD28CSD-CD3zSSD expressing Jurkat NFAT reporter CAR-T cells was used as reporter cells.

FIG. 6 depicts a diagnostic Jurkat NFAT reporter CAR-T cell assay using CD20 expressing SUDHDL4 tumor cells as target cells. A pool of anti-CD20-scFab-CD28ATD-CD28CSD-CD3zSSD expressing Jurkat NFAT reporter CAR-T cells was used as reporter cells.

FIG. 7 depicts a diagnostic Jurkat NFAT reporter CAR-T cell assay using CD20 expressing SUDHDL4 tumor cells as target cells. A pool of anti-CD20-Fab-CD28ATD-CD28CSD-CD3zSSD or anti-CD20-scFv-CD28ATD-CD28CSD-CD3zSSD expressing Jurkat NFAT reporter CAR-T cells was used as reporter cells.

FIG. 8A and FIG. 8B depict assessment of specificity of WT1/HLA-binders 5E11 and 33H09 by FACS with T2 cells pulsed with RMF-peptide or VLD-peptide.

FIG. 9 depicts activation of CAR-NFAT-signaling in a Jurkat NFAT reporter anti-WT1/HLA-Fab-CAR-T cell pool upon co-incubation with RMF- or VLD-peptide-pulsed T2 cells. Comparison of signals on RMF-peptide (target) vs. VLD-peptide (off-target) helps to assess specificity of activation.

FIG. 10 depicts activation of CAR-NFAT-signaling in Jurkat NFAT reporter anti-WT1/HLA-Fab-CAR-T cell pools upon co-incubation with RMF- or VLD-peptide-pulsed T2 cells. Comparison of signals on RMF-peptide (target) vs. VLD-peptide (off-target) helps to assess specificity of activation. Signals of NFAR reporter CAR-T cell pools incubated without target cells illustrate the low background of the assay.

FIG. 11 depicts activation of CAR-NFAT-signaling in Jurkat NFAT Fab-CAR-T cell pools expressing CAR-binders to different peptide/HLA-targets. Jurkat NFJAT CAR-T cell pools F06, F29 and F30 bind to a blinded peptide/HLA-target with an unrelated peptide.

FIG. 12 depicts a schematic representation of a diagnostic reporter CAR-T cell assay for detection of a MHC presented peptide.

FIG. 13 depicts a schematic representation of a diagnostic reporter CAR-T cell assay using anti-WT1/HLA-Fab-CAR transduced Jurkat NFAT reporter cells to detect WT1 positive cells in the bone marrow of an AML patient.

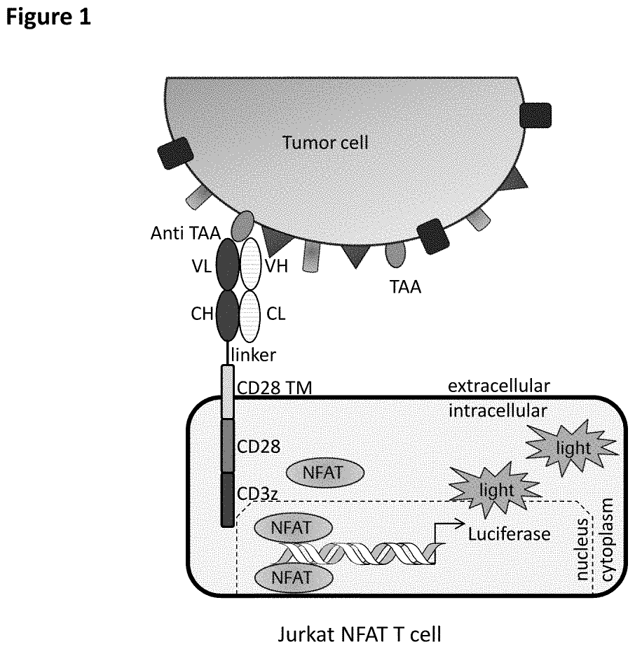

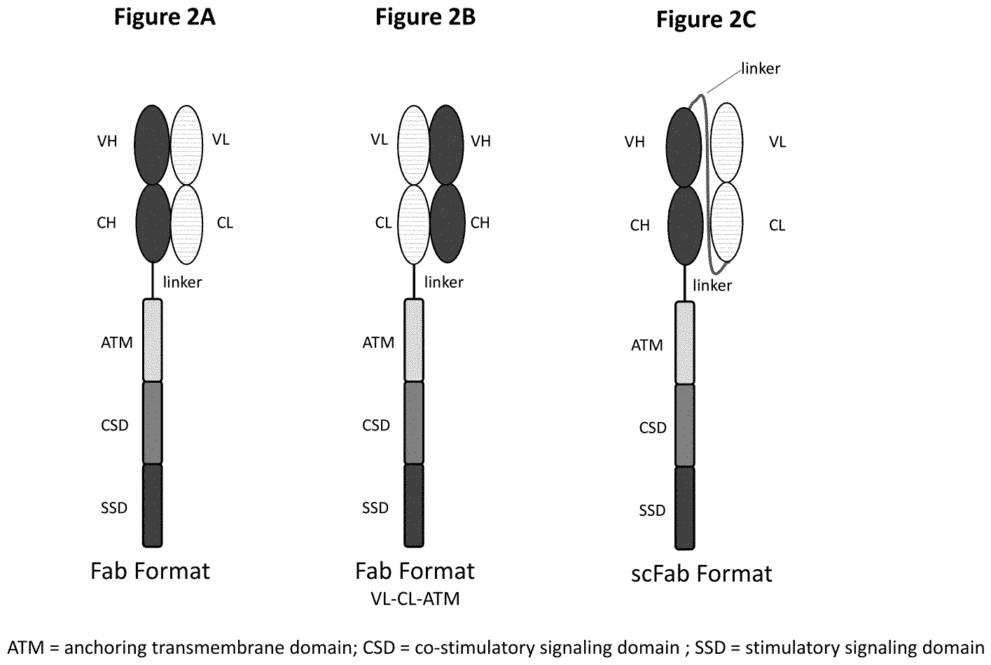

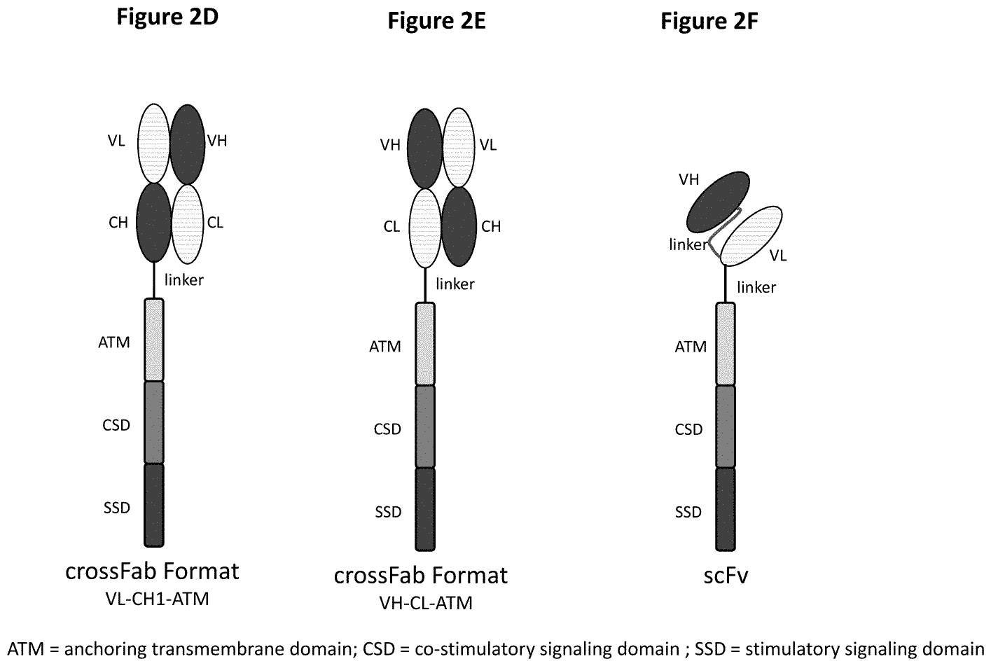

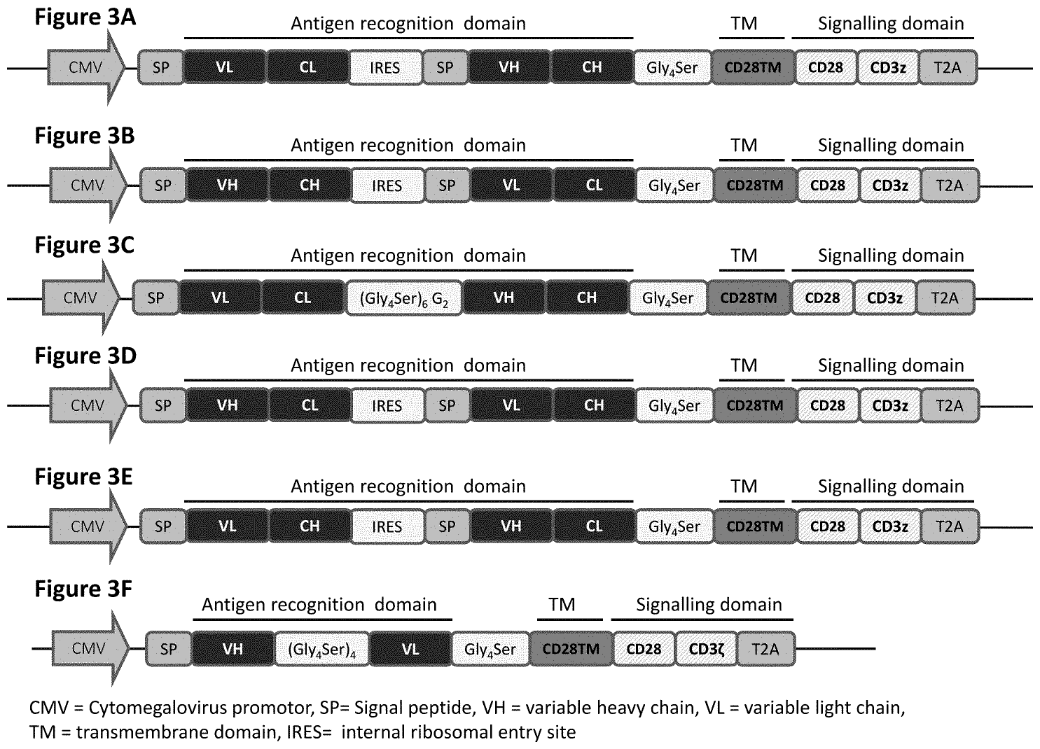

### DETAILED DESCRIPTION

“Affinity” refers to the strength of the sum total of non-covalent interactions between a single binding site of a molecule (e.g., an antibody or a CAR) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen and/or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A preferred method for measuring affinity is Surface Plasmon Resonance (SPR) and a preferred temperature for the measurement is 25° C.

The term “amino acid” (“aa”) refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics. Amino acid sequence deletions and insertions include amino- and/or carboxy-terminal deletions and insertions of amino acids. Particular amino acid mutations are amino acid substitutions. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

The term “antibody” herein is used in the broadest sense and encompasses various antibody structures, including but not limited to monoclonal antibodies, polyclonal antibodies, and antibody fragments so long as they exhibit the desired antigen-binding activity. Accordingly, in the context of the present invention, the term antibody relates to full immunoglobulin molecules as well as to parts of such immunoglobulin molecules. Furthermore, the term relates, as discussed herein, to modified and/or altered antibody molecules, in particular to modified antibody molecules. The term also relates to recombinantly or synthetically generated/synthesized antibodies. In the context of the present invention the term antibody is used interchangeably with the term immunoglobulin.

An “antibody fragment” refers to a molecule other than an intact antibody that comprises a portion of an intact antibody that binds the antigen to which the intact antibody binds. Examples of antibody fragments include but are not limited to Fv, Fab, crossover Fab, Fab′, Fab′-SH, F(ab′)₂, diabodies, linear antibodies, single-domain antibodies, single-chain antibody molecules (e.g., scFv, scFab), and single-domain antibodies. For a review of certain antibody fragments, see Hudson et al., Nat Med 9, 129-134 (2003). For a review of scFv fragments, see e.g., Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody (Domantis, Inc., Waltham, MA; see e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

As used herein, the term “antigen binding molecule” refers in its broadest sense to a molecule that specifically binds an antigenic determinant. Examples of antigen binding molecules are antibodies/immunoglobulins and derivatives, e.g., fragments, thereof. Furthermore, the term relates, as discussed herein, to modified and/or altered antigen binding molecules, in particular to modified antibody molecules. The term also relates to recombinantly or synthetically generated/synthesized antibodies. In the context of the present invention the antigen binding molecule is preferably an antibody or fragment thereof.

As used herein, the term “antigen binding moiety” refers to a polypeptide molecule that specifically binds to an antigenic determinant. In one embodiment, an antigen binding moiety is able to direct the entity to which it is attached (e.g., an immunoglobulin or a CAR) to a target site, for example to a specific type of tumor cell or tumor stroma bearing the antigenic determinant or to an immunoglobulin binding to the antigenic determinant on a tumor cell. In another embodiment an antigen binding moiety is able to activate signaling through its target antigen, for example signaling is activated upon binding of an antigenic determinant to a CAR on a T cell. In the context of the present invention, antigen binding moieties may be included in antibodies and fragments thereof as well as in antigen binding receptors (e.g., CARs) and fragments thereof as further defined herein. Antigen binding moieties include an antigen binding domain, e.g., comprising an immunoglobulin heavy chain variable region and an immunoglobulin light chain variable region.

In the context of the present invention the term “antigen binding receptor” relates to a molecule comprising an anchoring transmembrane domain and an extracellular domain comprising at least one antigen binding moiety. An antigen binding receptor (e.g., a CAR) can be made of polypeptide parts from different sources. Accordingly, it may be also understood as a “fusion protein” and/or a “chimeric protein”. Usually, fusion proteins are proteins created through the joining of two or more genes (or preferably cDNAs) that originally coded for separate proteins. Translation of this fusion gene (or fusion cDNA) results in a single polypeptide, preferably with functional properties derived from each of the original proteins. Recombinant fusion proteins are created artificially by recombinant DNA technology for use in biological research or therapeutics. In the context of the present invention a CAR (chimeric antigen receptor) is understood to be an antigen binding receptor comprising an extracellular portion comprising an antigen binding moiety fused by a spacer sequence to an anchoring transmembrane domain which is itself fused to the intracellular signaling domains of e.g., CD3z and CD28.

An “antigen binding site” refers to the site, i.e., one or more amino acid residues, of an antigen binding molecule which provides interaction with the antigen. A native immunoglobulin molecule typically has two antigen binding sites, a Fab or a scFv molecule typically has a single antigen binding site.

The term “antigen binding domain” refers to the part of an antibody or an antigen binding receptor (e.g., a CAR) that comprises the area which specifically binds to and is complementary to part or all of an antigen. An antigen binding domain may be provided by, for example, one or more immunoglobulin variable domains (also called variable regions). Particularly, an antigen binding domain comprises an immunoglobulin light chain variable region (VL) and an immunoglobulin heavy chain variable region (VH).

The term “variable region” or “variable domain” refers to the domain of an immunoglobulin heavy or light chain that is involved in binding the antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co, page 91 (2007). A single VH or VL domain is usually sufficient to confer antigen-binding specificity.

The term “ATD” as used herein refers to “anchoring transmembrane domain” which defines a polypeptide stretch capable of integrating in (the) cellular membrane(s) of a cell. The ATD can be fused to further extracellular and/or intracellular polypeptide domains wherein these extracellular and/or intracellular polypeptide domains will be confined to the cell membrane as well. In the context of the antigen binding receptors as used in the present invention the ATD confers membrane attachment and confinement of the antigen binding receptor, e.g., a CAR used according to the present invention.

The term “binding to” as used in the context of the antigen binding receptors (e.g., CARs) used according to the present invention defines a binding (interaction) of an “antigen-interaction-site” and an antigen with each other. The term “antigen-interaction-site” defines a motif of a polypeptide which shows the capacity of specific interaction with a specific antigen or a specific group of antigens. Said binding/interaction is also understood to define a “specific recognition”. The term “specifically recognizing” means in accordance with this invention that the antigen binding receptor is capable of specifically interacting with and/or binding to the recognition domain, i.e., a modified molecule as defined herein whereas the non-modified molecule is not recognized. The antigen binding moiety of an antigen binding receptor (e.g., a CAR) can recognize, interact and/or bind to different epitopes on the same molecule. This term relates to the specificity of the antigen binding receptor, i.e., to its ability to discriminate between the specific regions of a molecule. The specific interaction of the antigen-interaction-site with its specific antigen may result in an initiation of a signal, e.g., due to the induction of a change of the conformation of the polypeptide comprising the antigen, an oligomerization of the polypeptide comprising the antigen, an oligomerization of the antigen binding receptor, etc. Thus, a specific motif in the amino acid sequence of the antigen-interaction-site and the antigen bind to each other as a result of their primary, secondary or tertiary structure as well as the result of secondary modifications of said structure. The term binding to does not only relate to a linear epitope but may also relate to a conformational epitope, a structural epitope or a discontinuous epitope consisting of two regions of the target molecules or parts thereof. In the context of this invention, a conformational epitope is defined by two or more discrete amino acid sequences separated in the primary sequence which comes together on the surface of the molecule when the polypeptide folds to the native protein (Sela, Science 166 (1969), 1365 and Laver, Cell 61 (1990), 553-536). Moreover, the term “binding to” is interchangeably used in the context of the present invention with the term “interacting with”. The ability of the antigen binding moiety (e.g., a Fab or scFv domain) of a CAR or an antibody to bind to a specific target antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the target antigen as measured, in particular by SPR. In certain embodiments, an antigen binding moiety that binds to the target antigen, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M). An antigen binding moiety is said to “specifically bind” to a target antigen when the antigen binding moiety has a K_{D} of 1 μM or less and such interaction is herein referred to as “specific binding”. Accordingly, the antigen binding receptor (e.g., the CAR) used according to the invention specifically binds to/interacts with tumor target antigen. Cross-reactivity of a panel of constructs under investigation may be tested, for example, by assessing binding of a panel of antigen binding moieties under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the target antigen of interest. These methods may comprise, inter alia, binding studies, blocking and competition studies with structurally and/or functionally closely related domains. The binding studies also comprise FACS analysis, surface plasmon resonance (SPR, e.g., with BIAcore), analytical ultracentrifugation, isothermal titration calorimetry, fluorescence anisotropy, fluorescence spectroscopy or by radiolabeled ligand binding assays.

The term “CDR” as employed herein relates to “complementary determining region”, which is well known in the art. The CDRs are parts of immunoglobulins, antigen binding moieties and/or antigen binding receptors that determine the specificity of said molecules and make contact with a specific ligand. The CDRs are the most variable part of the molecule and contribute to the antigen binding diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in “Kabat” (Sequences of Proteins of Immunological Interest”, 5th edit. NIH Publication no. 91-3242 U.S. Department of Health and Human Services (1991); Chothia J. Mol. Biol. 196 (1987), 901-917) or “Chothia” (Nature 342 (1989), 877-883).

The term “ CD3z” refers to T-cell surface glycoprotein CD3 zeta chain, also known as “T-cell receptor T3 zeta chain” and “CD247”.

The term “chimeric antigen receptor” or “chimeric receptor” or “CAR” refers to an antigen binding receptor constituted of an extracellular portion of an antigen binding moiety (e.g., a scFv or a Fab) fused by a spacer sequence to intracellular signaling domains (e.g., of CD3z and CD28).

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called a, 6, E, y, and respectively.

By a “crossover Fab molecule” (also termed “crossFab” or “crossover Fab fragment”) is meant a Fab molecule wherein either the variable regions or the constant regions of the Fab heavy and light chain are exchanged, i.e., the crossFab fragment comprises a peptide chain composed of the light chain variable region and the heavy chain constant region, and a peptide chain composed of the heavy chain variable region and the light chain constant region. For clarity, in a crossFab fragment wherein the variable regions of the Fab light chain and the Fab heavy chain are exchanged, the peptide chain comprising the heavy chain constant region is referred to herein as the heavy chain of the crossover Fab molecule. Conversely, in a crossFab fragment wherein the constant regions of the Fab light chain and the Fab heavy chain are exchanged, the peptide chain comprising the heavy chain variable region is referred to herein as the heavy chain of the crossFab fragment. Accordingly, a crossFab fragment comprises a heavy or light chain composed of the heavy chain variable and the light chain constant regions (VH-CL), and a heavy or light chain composed of the light chain variable and the heavy chain constant regions (VL-CH1). In contrast thereto, by a “Fab” or “conventional Fab molecule” is meant a Fab molecule in its natural format, i.e., comprising a heavy chain composed of the heavy chain variable and constant regions (VH-CH1), and a light chain composed of the light chain variable and constant regions (VL-CL).

The term “CSD” as used herein refers to co-stimulatory signaling domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the terms “engineer”, “engineered”, “engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

A “Fab molecule” refers to a protein consisting of the VH and CH1 domain of the heavy chain (the “Fab heavy chain”) and the VL and CL domain of the light chain (the “Fab light chain”) of an antigen binding molecule.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the “EU numbering” system, also called the EU index, as described in Kabat et al., Sequences of Proteins of Immunological Interest, 5th Ed. Public Health Service, National Institutes of Health, Bethesda, Md., 1991. A subunit of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g., IgG 1 and IgG2), IgM, IgA, IgD, and IgE.) Antibodies can be from a single species e.g., human, or they can be chimerized or humanized antibodies. Full length antibodies usually comprise two antigen binding sites each formed by a pair of VH and VL, which both specifically bind to the same antigen. Furthermore, full length antibodies can comprise two antigen binding sites each formed by a pair of VH and VL, wherein the two antigen binding sites bind to different antigens, e.g., wherein the antibodies are bispecific. The C-terminus of the heavy or light chain of said full length antibody denotes the last amino acid at the C-terminus of said heavy or light chain.

By “fused” is meant that the components (e.g., a Fab and a transmembrane domain) are linked by peptide bonds, either directly or via one or more peptide linkers.

The terms “host cell”, “host cell line” and “host cell culture” are used interchangeably and refer to cells into which exogenous nucleic acid has been introduced, including the progeny of such cells. Host cells include “transformants” and “transformed cells” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate an antibody used according to the present invention. Host cells include cultured cells, e.g., mammalian cultured cells, such as CHO cells, BHK cells, NS0cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody and/or an antigen binding receptor or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of Kabat numbering to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antigen binding moiety variable region are according to the Kabat numbering system. The polypeptide sequences of the sequence listing are not numbered according to the Kabat numbering system. However, it is well within the ordinary skill of one in the art to convert the numbering of the sequences of the Sequence Listing to Kabat numbering.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human.

By “isolated nucleic acid” molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed below for polypeptides (e.g., ALIGN-2).

By an “isolated polypeptide” or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y; where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “nucleic acid molecule” relates to the sequence of bases comprising purine- and pyrimidine bases which are comprised by polynucleotides, whereby said bases represent the primary structure of a nucleic acid molecule. Herein, the term nucleic acid molecule includes DNA, cDNA, genomic DNA, RNA, synthetic forms of DNA and mixed polymers comprising two or more of these molecules. In addition, the term nucleic acid molecule includes both, sense and antisense strands. Moreover, the herein described nucleic acid molecule may contain non-natural or derivatized nucleotide bases, as will be readily appreciated by those skilled in the art.

As used herein “NFAT” refers to the “nuclear factor of activated T-cells” and is a family of transcription factors which is expressed in most immune cells. Activation of transcription factors of the NFAT family is dependent on calcium signaling. As an example, T cell activation through the T cell synapse results in calcium influx. Increased intracellular calcium levels activate the calcium-sensitive phosphatase, calcineurin, which rapidly dephosphorylates the serine-rich region (SRR) and SP-repeats in the amino termini of NFAT proteins. This results in a conformational change that exposes a nuclear localization signal promoting NFAT nuclear import and activation of target genes.

As used herein “NFAT pathway” refers to the stimuli that lead to modulation of activity of member of the NFAT family of transcription factors. NFAT DNA elements are known to the art and are herein also referred to as “response element of the NFAT pathway”. Hence, a “receptor of the NFAT pathway” refers to a receptor which can trigger the modulation of activity of NFAT. Examples of a “receptor of the NFAT pathway” are e.g., T cell receptor and B cell receptor.

As used herein “NF-κB” refers to the “nuclear factor kappa-light-chain-enhancer of activated B cells” and is a transcription factor which is implicated in the regulation of many genes that code for mediators of apoptosis, viral replication, tumorigenesis, various autoimmune diseases and inflammatory responses. NFκB is present in almost all eukaryotic cells. Generally, it is located in the cytosol in an inactive state, since it forms a complex with inhibitory kappa B (IκB) proteins. Through the binding of ligands to integral membrane receptors (also referred to as “receptors of the NF-κB pathway”, the IκB kinase (IKK) is activated. IKK is an enzyme complex which consists of two kinases and a regulatory subunit. This complex phosphorylates the IκB proteins, which leads to ubiquitination and therefore degradation of those proteins by the proteasome. Finally, the free NFκB is in an active state, translocates to the nucleus and binds to the κB DNA elements and induces transcription of target genes.

As used herein “NF-κB pathway” refers to the stimuli that lead to modulation of activity of NF-κB. For example activation of the Toll-like receptor signaling, TNF receptor signaling, T cell receptor and B cell receptor signaling through either binding of a ligand or an antibody result in activation of NF-κB. Subsequently, phosphorylated NF-κB dimers bind to κB DNA elements and induce transcription of target genes. κB DNA elements are known in the art and herein also referred to as “response element of the NF-κB pathway”. Hence, a “receptor of the NF-κB pathway” refers to a receptor which can trigger the modulation of activity of NF-κB. Examples of a “receptor of the NF-κB pathway” are Toll-like receptors, TNF receptors, T cell receptor and B cell receptor.

As used herein “AP-1” refers to the “activator protein 1” and is a transcription factor which is involved a number of cellular processes including differentiation, proliferation, and apoptosis. AP-1 functions are dependent on the specific Fos and Jun subunits contributing to AP-1 dimers. AP-1 binds to a palindromic DNA motif (5′-TGA G/C TCA-3′) to regulate gene expression.

The term “pharmaceutical composition” refers to a preparation which is in such form as to permit the biological activity of an active ingredient contained therein to be effective, and which contains no additional components which are unacceptably toxic to a subject to which the formulation would be administered. A pharmaceutical composition usually comprises one or more pharmaceutically acceptable carrier(s).

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, the term “polypeptide” refers to a molecule composed of monomers (amino acids) linearly linked by amide bonds (also known as peptide bonds). The term polypeptide refers to any chain of two or more amino acids, and does not refer to a specific length of the product. Thus, peptides, dipeptides, tripeptides, oligopeptides, protein, amino acid chain, or any other term used to refer to a chain of two or more amino acids, are included within the definition of polypeptide, and the term polypeptide may be used instead of, or interchangeably with any of these terms. The term polypeptide is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term nucleic acid molecule refers to any one or more nucleic acid segments, e.g., DNA or RNA fragments, present in a polynucleotide.

The term “protein with intrinsic fluorescence” refers to a protein capable of forming a highly fluorescent, intrinsic chromophore either through the cyclization and oxidation of internal amino acids within the protein or via the enzymatic addition of a fluorescent co-factor. The term “protein with intrinsic fluorescence” includes wild-type fluorescent proteins and mutants that exhibit altered spectral or physical properties. The term does not include proteins that exhibit weak fluorescence by virtue only of the fluorescence contribution of non-modified tyrosine, tryptophan, histidine and phenylalanine groups within the protein. Proteins with intrinsic fluorescence are known in the art, e.g., green fluorescent protein (GFP),), red fluorescent protein (RFP), Blue fluorescent protein (BFP, Heim et al. 1994, 1996), a cyan fluorescent variant known as CFP (Heim et al. 1996; Tsien 1998); a yellow fluorescent variant known as YFP (Oruro et al. 1996; Wachter et al. 1998); a violet-excitable green fluorescent variant known as Sapphire (Tsien 1998; Zapata-Hommer et al. 2003); and a cyan-excitable green fluorescing variant known as enhanced green fluorescent protein or EGFP (Yang et al. 1996) and can be measured e.g., by live cell imaging (e.g., Incucyte) or fluorescent spectrophotometry.

“Reduced binding” refers to a decrease in affinity for the respective interaction, as measured for example by SPR. For clarity the term includes also reduction of the affinity to zero (or below the detection limit of the analytic method), i.e., complete abolishment of the interaction. Conversely, “increased binding” refers to an increase in binding affinity for the respective interaction.

The term “regulatory sequence” refers to DNA sequences, which are necessary to effect the expression of coding sequences to which they are ligated. The nature of such control sequences differs depending upon the organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

As used herein, a “reporter gene” means a gene whose expression can be assayed. In one preferred embodiment a “reporter gene” is a gene that encodes a protein the production and detection of which is used as a surrogate to detect indirectly the activity of the antibody or ligand to be tested. The reporter protein is the protein encoded by the reporter gene. Preferably, the reporter gene encodes an enzyme whose catalytic activity can be detected by a simple assay method or a protein with a property such as intrinsic fluorescence or luminescence so that expression of the reporter gene can be detected in a simple and rapid assay requiring minimal sample preparation. Non-limiting examples of enzymes whose catalytic activity can be detected are Luciferase, beta Galactosidase, Alkaline Phosphatase. Luciferase is a monomeric enzyme with a molecular weight (MW) of 61 kDa. It acts as a catalysator and is able to convert D-luciferin in the presence of Adenosine triphosphate (ATP) and Mg2+ to luciferyl adenylate. In addition, pyrophosphate (PPi) and adenosine monophosphate (AMP) are generated as byproducts. The intermediate luciferyl adenylate is then oxidized to oxyluciferin, carbon dioxide (CO₂) and light. Oxyluciferin is a bioluminescent product which can be quantitatively measured in a luminometer by the light released from the reaction. Luciferase reporter assays are commercially available and known in the art, e.g., Luciferase 1000 Assay System and ONE-Glo™ Luciferase Assay System.

A “response element” refers to a specific transcription factor binding element, or cis acting element which can be activated or silenced on binding of a certain transcription factor. In one embodiment the response element is a cis-acting enhancer element located upstream of a minimal promotor (e.g., a TATA box promotor) which drives expression of the reporter gene upon transcription factor binding.

As used herein, the term “single-chain” refers to a molecule comprising amino acid monomers linearly linked by peptide bonds. In certain embodiments, one of the antigen binding moieties is a scFv fragment, i.e., a VH domain and a VL domain connected by a peptide linker. In certain embodiments, one of the antigen binding moieties is a single-chain Fab molecule, i.e., a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

The term “SSD” as used herein refers to stimulatory signaling domain.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

In the context of the present invention, the term tag” refers to a molecule attached or engrafted to or onto a biomolecule such as a protein, particularly an antigen binding molecule.

The function of a tag is to mark or label the “tagged” protein (e.g., an immunoglobulin or fragment thereof) such that it can be recognized by a specific antigen binding moiety capable of binding to the tag but not capable of binding to the untagged protein. The term is synonymous to “molecular tag” and comprises without being limited to fluorescent tags, protein tags, affinity tags, solubilization tags, chromatography tags, epitope tags and small molecule tags such as hapten tags. Small molecule tags, e.g., haptens, can be chemically coupled covalently or non-covalently to the biomolecule whereas “protein tags” or “polypeptide tags” are peptide sequences which can be genetically grafted onto a protein and subsequently be recognized by specific antigen binding moieties capable of binding to the tag but not capable of binding to the untagged protein. Hapten tags are able to elicit an immune response when attached to a carrier protein, and, therefore, are suitable to generate specific antigen binding moieties capable of recognizing the tag on a carrier such as a protein. In preferred embodiments of the present invention, the tag is a hapten tag or a polypeptide tag.

As used herein, the term “target antigenic determinant” is synonymous with “target antigen”, “target epitope” and “target cell antigen” and refers to a site (e.g., a contiguous stretch of amino acids or a conformational configuration made up of different regions of non-contiguous amino acids) on a polypeptide macromolecule to which an antibody binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g., CD20, CD38, CD138, CEA, EGFR, Fo1R1, HER2, LeY, MCSP, STEAP1, TYRP1, and WT1) can be any native form of the proteins from any vertebrate source, including mammals such as primates (e.g., humans) and rodents (e.g., mice and rats), unless otherwise indicated. In a particular embodiment the target antigen is a human protein. Where reference is made to a specific target protein herein, the term encompasses the “full-length”, unprocessed target protein as well as any form of the target protein that results from processing in the target cell. The term also encompasses naturally occurring variants of the target protein, e.g., splice variants or allelic variants. Exemplary human target proteins useful as antigens include, but are not limited to: CD20, CD38, CD138, CEA, EGFR, Fo1R1, HER2, LeY, MCSP, STEAP1, TYRP1, and WT1.

Antibodies may have one, two, three or more binding domains and may be monospecific, bispecific or multispecific. The antibodies can be full length from a single species, or be chimerized or humanized. For an antibody with more than two antigen binding domains, some binding domains may be identical and/or have the same specificity.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. Suitable assays to measure T cell activation are known in the art and described herein.

In accordance with this invention, the term “T cell receptor” or “TCR” is commonly known in the art. In particular, herein the term “T cell receptor” refers to any T cell receptor, provided that the following three criteria are fulfilled: (i) tumor specificity, (ii) recognition of (most) tumor cells, which means that an antigen or target should be expressed in (most) tumor cells and (iii) that the TCR matches to the HLA-type of the subjected to be treated. In this context, suitable T cell receptors which fulfill the above mentioned three criteria are known in the art such as receptors recognizing NY-ESO-1 (for sequence information(s) see, e.g., PCT/GB2005/001924) and/or HER2neu (for sequence information(s) see WO-Al 2011/0280894). Major histocompatibility complex (MHC) class I molecules present peptides from endogenous antigens to CD8+ cytotoxic T cells, and therefore, MHC-peptide complexes are a suitable target for immunotherapeutic approaches. The MHC-peptide complexes can be targeted by recombinant T-cell receptors (TCRs). However, most TCRs may have affinities which are too low for immunotherapy whereas high affinity binding moieties with TCR specificity would be beneficial. Towards this end, high-affinity soluble antibody molecules with TCR-like specificity can be generated, e.g., by generating phage display libraries (e.g., combinatorial libraries) and screening such libraries as further described herein. These soluble antigen binding moieties e.g., scFv or Fab, with TCR-like specificity as described herein are referred to as “T cell receptor like antigen binding moieties” or “TCRL antigen binding moieties”.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode antigen binding receptors of the invention or fragments thereof.

In this context, provided herein are diagnostic methods, particularly in vitro methods, for detecting cells, in particular tumor cells, in a sample. In a preferred embodiment, the sample is a patient sample, e.g., deriving from a biopsy or a body fluid in which aberrant cells need to be detected. The assays of the present invention combine the high specificity of chimeric antigen receptors (CARs) comprising antigen binding moieties, in particular scFv and/or Fab fragments, with the sensitivity of luminescence detection of a reporter signal. In the herein described methods and assays, the target antigen binding moiety mediates the contact between a target cell, in particular a cancer cell, and a reporter cell, in particular a T cell, e.g., a Jurkat cell. In this context, the methods as described herein are useful to detect a cancer cell according to specificity of binding of a CAR introduced in a suitable reporter cells, preferably a reporter T cell, e.g, a Jurkat cell.

Accordingly, in one embodiment, provided is a diagnostic assay for determining the presence of a tumor cell in a sample, the diagnostic assay comprising the steps of:
a) contacting the sample with a chimeric antigen receptor (CAR) expressing reporter T (CAR-T) cell wherein the reporter CAR-T cell comprises:
   i. a CAR capable of specific interaction with the tumor cell, wherein the CAR is operationally coupled to a response element;
   ii. a reporter gene under the control of the response element; and
b) determining T cell activation by measuring the expression of the reporter gene to establish the presence of the tumor cell.

Further provided and used for the present invention are transduced T cells capable of expression of the herein described CAR molecule(s). The transduced T cells comprise a reporter gene under the control of a response element, wherein the CAR is operationally coupled to the response element as herein described. Upon binding of the target antigen binding moiety to the target cell, e.g., the tumor cell, the reporter CAR-T cell, e.g., the Jurkat cell, becomes activated and the reporter gene is expressed. Expression of the reporter gene is therefore indicative for (specific) binding of the CAR in the context of T cell activation induced by binding of a T cell to a target cell, e.g., on a tumor cell.

In this context further described and used for the methods of the present invention are CARs capable of specific binding to a tumor target antigen. In one embodiment, the CAR comprises a target antigen binding moiety capable of specific binding to a tumor target antigen. Examples of suitable tumor targets are proteins exclusively or mainly expressed on the surface of tumor cells, such as for example but not limited to CD20, CD38, CD138, CEA, EGFR, Fo1R1, HER2, LeY, MCSP, STEAP1, TYRP1, and WT1, or fragments thereof.

The present invention further describes the transduction and use of T cells, such as CD8+ T cells, CD4+ T cells, CD3+ T cells, λδ T cells or natural killer (NK) T cells and immortalized cell lines, e.g., Jurkat cells, to introduce a reporter system as described herein and (a) CAR(s) as described herein and their targeted recruitment and activation mediated by the herein described CAR which is capable of direct binding to a target antigen on the surface of the target cell, e.g., on the surface of a tumor cell.

Accordingly, the invention provides a versatile diagnostic platform wherein CARs may be used as a specific guidance for immune cells, in particular wherein T cells are specifically targeted toward the tumor cells by the CARs as described herein. After engagement of the CAR to the target antigen on the surface of a tumor cell, the reporter cell becomes activated wherein the activation can be measured, e.g., by read-out of a fluorescent or luminescent signal. The platform is flexible and specific by allowing the use of diverse existing or newly developed target antigen binding moieties.

Antigen binding moieties capable of specific binding to a target antigen, e.g., a tumor antigen, may be generated by immunization of e.g., a mammalian immune system. Such methods are known in the art and e.g., are described in Burns in Methods in Molecular Biology 295:1-12 (2005). Alternatively, antigen binding moieties of desired activity may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antigen binding moieties possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O′Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992) and in Marks and Bradbury in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antigen binding moieties to the immunogen without the requirement of constructing hybridomas. Alternatively, the naive repertoire can be cloned (e.g., from human) to provide a single source of antigen binding moieties to a wide range of non-self and also self antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764, 2007/0292936 and 2009/0002360. Further examples of methods known in the art for screening combinatorial libraries for antigen binding moieties with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

In a first illustrative embodiment of the present invention, as a proof of concept, provided is the use of a reporter cell, e.g., a Jurkat cell expressing a CAR capable of specific binding to target antigen human CD20.

In one embodiment the CAR capable of specific binding to CD20 comprises the heavy chain complementarity determining regions (CDRs) of SEQ ID NO:1, SEQ ID NO:2 and SEQ ID NO:3 and the light chain CDRs of SEQ ID NO:4, SEQ ID NO:5 and SEQ ID NO:6. In one preferred embodiment the CAR capable of specific binding to CD20 comprises a heavy chain variable region comprising:
(a) a heavy chain complementarity determining region (CDR H) 1 amino acid sequence of YSWIN (SEQ ID NO:1);
(b) a CDR H2 amino acid sequence of RIFPGDGDTDYNGKFKG (SEQ ID NO:2);
(c) a CDR H3 amino acid sequence of NVFDGYWLVY (SEQ ID NO:3); and a light chain variable region comprising:
(d) a light chain (CDR L)1 amino acid sequence of RSSKSLLHSNGITYLY (SEQ ID NO:4);
(e) a CDR L2 amino acid sequence of QMSNLVS (SEQ ID NO:5); and
(f) a CDR L3 amino acid sequence of AQNLELPYT (SEQ ID NO:6).

In one embodiment the CAR capable of specific binding to CD20 comprises a heavy chain variable region (VH) comprising an amino acid sequence that is at least about 95%, 96%, 97%, 98%, 99% or 100% identical to the amino acid sequence of SEQ ID NO:12 and a light chain variable region (VL) comprising an amino acid sequence that is at least about 95%, 96%, 97%, 98%, 99% or 100% identical to the amino acid sequence of SEQ ID NO:10.

In one embodiment the CAR capable of specific binding to CD20 comprises a heavy chain variable region (VH) comprising the amino acid sequence of SEQ ID NO:12, and a light chain variable region (VL) comprising the amino acid sequence of SEQ ID NO:10.

In one embodiment, the at least one antigen binding moiety is a scFv, a Fab, a crossFab or a scFab fragment. In one embodiment the CAR capable of specific binding to CD20 comprises a Fab fragment. In a preferred embodiment the CAR capable of specific binding to CD20 comprises a Fab fragment comprising a heavy chain of SEQ ID NO:8 and a light chain of SEQ ID NO:9 .

In one embodiment the antigen binding moiety capable of specific binding to CD20 is a Fab fragment comprising a heavy chain comprising or consisting of an amino acid sequence of SEQ ID NO:8 and a light chain comprising or consisting of the amino acid sequence of SEQ ID NO:9.

In a particular embodiment, the antigen binding moiety is a Fab fragment capable of specific binding to CD20, wherein the antigen binding receptor comprises a heavy chain fusion polypeptide comprising an amino acid sequence that is at least about 95%, 96%, 97%, 98%, 99% or 100% identical to the amino acid sequence of SEQ ID NO:7 and a light chain polypeptide comprising an amino acid sequence that is at least about 95%, 96%, 97%, 98%, 99% or 100% identical to the amino acid sequence of SEQ ID NO:9.

In a preferred embodiment, the antigen binding moiety is a Fab fragment capable of specific binding to CD20, wherein the antigen binding receptor comprises a heavy chain fusion polypeptide comprising the amino acid sequence of SEQ ID NO:7 and a light chain polypeptide comprising the amino acid sequence of SEQ ID NO:9.

In one embodiment the CAR capable of specific binding to CD20 comprises a scFv fragment which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv fragment has the configuration VH-linker-VL.

In another illustrative embodiment of the present invention, as a proof of concept, provided is the use of a reporter cell, e.g., a Jurkat cell expressing a CAR capable of specific binding to the human carcinoembryonic antigen (CEA).

In one embodiment the CAR capable of specific binding to CEA comprises the heavy chain complementarity determining regions (CDRs) of SEQ ID NO:36, SEQ ID NO:37 and SEQ ID NO:38 and the light chain CDRs of SEQ ID NO:39, SEQ ID NO:40 and SEQ ID NO:41.

In one preferred embodiment the CAR capable of specific binding to CEA comprises a heavy chain variable region comprising:
(a) a heavy chain complementarity determining region (CDR H) 1 amino acid sequence of EFGMN (SEQ ID NO:36);
(b) a CDR H2 amino acid sequence of WINTKTGEATYVEEFKG (SEQ ID NO:37);
(c) a CDR H3 amino acid sequence of WDFAYYVEAMDY (SEQ ID NO:38);
   and a light chain variable region comprising:
(d) a light chain (CDR L)1 amino acid sequence of KASAAVGTYVA (SEQ ID NO:39);
(e) a CDR L2 amino acid sequence of SASYRKR (SEQ ID NO:40); and
(f) a CDR L3 amino acid sequence of HQYYTYPLFT (SEQ ID NO:41).

In one embodiment the CAR capable of specific binding to CEA comprises a heavy chain variable region (VH) comprising an amino acid sequence that is at least about 95%, 96%, 97%, 98%, 99% or 100% identical to the amino acid sequence of SEQ ID NO:44 and a light chain variable region (VL) comprising an amino acid sequence that is at least about 95%, 96%, 97%, 98%, 99% or 100% identical to the amino acid sequence of SEQ ID NO:45.

In one embodiment the CAR capable of specific binding to CEA comprises a heavy chain variable region (VH) comprising the amino acid sequence of SEQ ID NO: 44, and a light chain variable region (VL) comprising the amino acid sequence of SEQ ID NO: 45.

In one embodiment, the at least one antigen binding moiety is a scFv, a Fab, a crossFab or a scFab fragment. In one embodiment the CAR capable of specific binding to CEA comprises a scFv fragment which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv fragment has the configuration VH-linker-VL.

In a preferred embodiment the CAR capable of specific binding to CEA comprises an scFv fragment comprising the amino acid sequence of SEQ ID NO:43.

In another illustrative embodiment of the present invention, as a proof of concept, provided is the use of a reporter cell, e.g., a Jurkat cell expressing a CARs capable of specific binding to a peptide/MHC complex wherein the peptide derives from human Wilms tumor 1 (WT1).

In one embodiment the CAR capable of specific binding to WT1 peptide/MHC complex comprises the heavy chain complementarity determining regions (CDRs) of SEQ ID NO:46, SEQ ID NO:47 and SEQ ID NO:48 and the light chain CDRs of SEQ ID NO:49, SEQ ID NO:50 and SEQ ID NO:51.

In another embodiment the CAR capable of specific binding to WT1 peptide/MHC complex comprises the heavy chain complementarity determining regions (CDRs) of SEQ ID NO:46, SEQ ID NO:47 and SEQ ID NO:57 and the light chain CDRs of SEQ ID NO:49, SEQ ID NO:50 and SEQ ID NO:58.

In one preferred embodiment the CAR capable of specific binding to WT1 peptide/MHC complex comprises a heavy chain variable region comprising:
(a) a heavy chain complementarity determining region (CDR H) 1 amino acid sequence of GGTFSSYAIS (SEQ ID NO:46);
(b) a CDR H2 amino acid sequence of GIIPIFGTANYAQKFQG (SEQ ID NO:47);
(c) a CDR H3 amino acid sequence selected from the group consisting of SIELWWGGFDY (SEQ ID NO:48) and GSYDLFSLDY (SEQ ID NO:57);
   and a light chain variable region comprising:
(d) a light chain (CDR L)1 amino acid sequence of RASQSISSWLA (SEQ ID NO:49);
(e) a CDR L2 amino acid sequence of DASSLES (SEQ ID NO:50); and
(f) a CDR L3 amino acid sequence selected from the group consisting of QQYEDYTT (SEQ ID NO:51) and QQYYDGIT (SEQ ID NO:58).

In one specific embodiment the CAR capable of specific binding to WT1 peptide/MHC complex comprises a heavy chain variable region comprising:
(g) a heavy chain complementarity determining region (CDR H) 1 amino acid sequence of GGTFSSYAIS (SEQ ID NO:46);
(h) a CDR H2 amino acid sequence of GIIPIFGTANYAQKFQG (SEQ ID NO:47);
(i) a CDR H3 amino acid sequence of SIELWWGGFDY (SEQ ID NO:48); and a light chain variable region comprising:
(j) a light chain (CDR L)1 amino acid sequence of RASQSISSWLA (SEQ ID NO:49);
(k) a CDR L2 amino acid sequence of DASSLES (SEQ ID NO:50); and
(l) a CDR L3 amino acid sequence of QQYEDYTT (SEQ ID NO:51).
   In another specific embodiment the CAR capable of specific binding to WT1 peptide/MHC complex comprises a heavy chain variable region comprising:
(a) a heavy chain complementarity determining region (CDR H) 1 amino acid sequence of GGTFSSYAIS (SEQ ID NO:46);
(b) a CDR H2 amino acid sequence of GIIPIFGTANYAQKFQG (SEQ ID NO:47);
(c) a CDR H3 amino acid sequence of GSYDLFSLDY (SEQ ID NO:57);
   and a light chain variable region comprising:
(d) a light chain (CDR L)1 amino acid sequence of RASQSISSWLA (SEQ ID NO:49);
(e) a CDR L2 amino acid sequence of DASSLES (SEQ ID NO:50); and
(f) a CDR L3 amino acid sequence of QQYYDGIT (SEQ ID NO:58).

In one embodiment the CAR capable of specific binding to WT1 peptide/MHC complex comprises a heavy chain variable region (VH) comprising an amino acid sequence that is at least about 95%, 96%, 97%, 98%, 99% or 100% identical to an amino acid sequence selected from the group consisting of SEQ ID NO:56 and SEQ ID NO:61 and a light chain variable region (VL) comprising an amino acid sequence that is at least about 95%, 96%, 97%, 98%, 99% or 100% identical to an amino acid sequence selected from the group consisting of SEQ ID NO:55 and SEQ ID NO:62.

In one embodiment the CAR capable of specific binding to WT1 peptide/MHC complex comprises a heavy chain variable region (VH) comprising the amino acid sequence of SEQ ID NO: 56, and a light chain variable region (VL) comprising the amino acid sequence of SEQ ID NO: 55.

In one embodiment the CAR capable of specific binding to WT1 peptide/MHC complex comprises a heavy chain variable region (VH) comprising the amino acid sequence of SEQ ID NO: 61, and a light chain variable region (VL) comprising the amino acid sequence of SEQ ID NO: 62.

In one embodiment, the at least one antigen binding moiety is a scFv, a Fab, a crossFab or a scFab fragment.

In a particular embodiment, the antigen binding moiety is a Fab fragment capable of specific binding to WT1 peptide/MHC complex, wherein the antigen binding receptor comprises a heavy chain fusion polypeptide comprising an amino acid sequence that is at least about 95%, 96%, 97%, 98%, 99% or 100% identical to the amino acid sequence of SEQ ID NO:52 and a light chain polypeptide comprising an amino acid sequence that is at least about 95%, 96%, 97%, 98%, 99% or 100% identical to the amino acid sequence of SEQ ID NO:54.

In a preferred embodiment, the antigen binding moiety is a Fab fragment capable of specific binding to WT1 peptide/MHC complex, wherein the antigen binding receptor comprises a heavy chain fusion polypeptide comprising the amino acid sequence of SEQ ID NO:52 and a light chain polypeptide comprising the amino acid sequence of SEQ ID NO:54.

In one embodiment the CAR capable of specific binding to WT1 peptide/MHC complex comprises a scFv fragment which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv fragment has the configuration VH-linker-VL.

In a preferred embodiment the CAR capable of specific binding to WT1 peptide/MHC comprises an scFv fragment comprising the amino acid sequence of SEQ ID NO:60.

In a particular embodiment, CAR capable of specific binding to WT1 peptide/MHC comprises an amino acid sequence that is at least about 95%, 96%, 97%, 98%, 99% or 100% identical to the amino acid sequence of: SEQ ID NO:59.

In a preferred embodiment, the CARcapable of specific binding to WT1 peptide/MHC comprises the amino acid sequence of SEQ ID NO:59.

Antigen binding moieties comprising a heavy chain variable domain (VH) and a light chain variable domain (VL), such as the Fab, crossFab, scFv and scFab fragments as described herein might be further stabilized by introducing interchain disulfide bridges between the VH and the VL domain. Accordingly, in one embodiment, the Fab fragment(s), the crossFab fragment(s), the scFv fragment(s) and/or the scFab fragment(s) comprised in the antigen binding receptors according to the invention might be further stabilized by generation of interchain disulfide bonds via insertion of cysteine residues (e.g., position 44 in the variable heavy chain and position 100 in the variable light chain according to Kabat numbering). Such stabilized antigen binding moieties are herein referred to by the term “ds”.

The CARs as provided and used herein comprise an extracellular domain comprising an antigen binding moiety capable of specific binding to the target antigen, an anchoring transmembrane domain and at least one intracellular signaling and/or at least one co-stimulatory signaling domain. The anchoring transmembrane domain mediates confinement of the CAR to the cell membrane of the reporter cell, e.g., the Jurkat cell. The intracellular signaling and/or at least one co-stimulatory signaling domain transfer the binding of the CAR to an intracellular signal, e.g., T cell activation, which can be assessed by measuring reporter gene expression. In the context of the present invention, expression of the reporter gene as described herein is indicative for binding of the target antigen binding moiety to the target antigen and resulting T cell activation as described herein.

The anchoring transmembrane domain of the CAR may be characterized by not having a cleavage site for mammalian proteases. Proteases refer to proteolytic enzymes that are able to hydrolyze the amino acid sequence of a transmembrane domain comprising a cleavage site for the protease. The term proteases include both endopeptidases and exopeptidases. In the context of the present invention any anchoring transmembrane domain of a transmembrane protein as laid down among others by the CD-nomenclature may be used to generate a CAR suitable according to the invention, which activates T cells, upon binding to a target cell, as herein described.

Accordingly, in the context of the present invention, the anchoring transmembrane domain may comprise part of a murine/mouse or preferably of a human transmembrane domain. An example for such an anchoring transmembrane domain is a transmembrane domain of CD28, for example, having the amino acid sequence as shown herein in SEQ ID NO:14 (as encoded by the DNA sequence shown in SEQ ID NO:29). In the context of the present invention, the transmembrane domain of the CAR may comprise/consist of an amino acid sequence as shown in SEQ ID NO:14 (as encoded by the DNA sequence shown in SEQ ID NO:29).

Alternatively, any protein having a transmembrane domain, as provided among others by the CD nomenclature, may be used as an anchoring transmembrane domain of the CAR provided and used in the invention. As described above, the CAR may comprise the anchoring transmembrane domain of CD28 which is located at amino acids 153 to 179, 154 to 179, 155 to 179, 156 to 179, 157 to 179, 158 to 179, 159 to 179, 160 to 179, 161 to 179, 162 to 179, 163 to 179, 164 to 179, 165 to 179, 166 to 179, 167 to 179, 168 to 179, 169 to 179, 170 to 179, 171 to 179, 172 to 179, 173 to 179, 174 to 179, 175 to 179, 176 to 179, 177 to 179 or 178 to 179 of the human full length CD28 protein as shown in SEQ ID NO:68 (as encoded by the cDNA shown in SEQ ID NO:67). Accordingly, in context of the present invention the anchoring transmembrane domain may comprise or consist of an amino acid sequence as shown in SEQ ID NO:14 (as encoded by the DNA sequence shown in SEQ ID NO:29).

As described herein, the CAR used according to the invention comprises at least one stimulatory signaling and/or co-stimulatory signaling domain. The stimulatory signaling and/or co-stimulatory signaling domain transduce the binding of the CAR to the tumor target antigen to an intracellular signal in the reporter CAR-T cell (e.g., the Jurkat cell). Accordingly, the CAR preferably comprises a stimulatory signaling domain, which provides T cell activation. In a preferred embodiment, binding of the target antigen binding moiety to the target leads to activation of the intracellular signaling and/or co-signaling domain. In certain embodiments, the herein provided CAR comprises a stimulatory signaling domain which is a fragment/polypeptide part of murine/mouse or human CD3z (the UniProt Entry of the human CD3z is P20963 (version number 177 with sequence number 2; the UniProt Entry of the murine/mouse CD3z is P24161 (primary citable accession number) or Q9D3G3 (secondary citable accession number) with the version number 143 and the sequence number 1)), FCGR3A (the UniProt Entry of the human FCGR3A is P08637 (version number 178 with sequence number 2)), or NKG2D (the UniProt Entry of the human NKG2D is P26718 (version number 151 with sequence number 1); the UniProt Entry of the murine/mouse NKG2D is 054709 (version number 132 with sequence number 2)). Thus, the stimulatory signaling domain which is comprised in the CAR may be a fragment/polypeptide part of the full length of CD3z, FCGR3A or NKG2D. The amino acid sequence of the murine/mouse full length of CD3z is shown herein as SEQ ID NO:65 (murine/mouse as encoded by the DNA sequence shown in SEQ ID NO:66). The amino acid sequence of the human full length CD3z is shown herein as SEQ ID NO:63 (human as encoded by the DNA sequence shown in SEQ ID NO:64). The CAR provided and used according to the present invention may comprise fragments of CD3z, FCGR3A or NKG2D as stimulatory domain, provided that at least one signaling domain is comprised. In particular, any part/fragment of CD3z, FCGR3A, or NKG2D is suitable as stimulatory domain as long as at least one signaling motive is comprised. However, more preferably, the CAR comprises polypeptides which are derived from human origin. Preferably, the CAR comprises the amino acid sequence as shown herein as SEQ ID NO:63 (CD3z) (human as encoded by the DNA sequences shown in SEQ ID NO:64 (CD3z)). For example, the fragment/polypeptide part of the human CD3z which may be comprised in the CAR may comprise or consist of the amino acid sequence shown in SEQ ID NO:16 (as encoded by the DNA sequence shown in SEQ ID NO:31). Accordingly, in one embodiment the CAR comprises the sequence as shown in SEQ ID NO:16 or a sequence which has up to 1, 2, 3, 4, 5, 6, 7, 8, 9, 10, 11, 12, 13, 14, 15, 16, 17, 18, 19, 20, 21, 22, 23, 23, 24, 25, 26, 27, 28, 29 or 30 substitutions, deletions or insertions in comparison to SEQ ID NO:16 and which is characterized by having a stimulatory signaling activity. Specific configurations of CARs comprising a stimulatory signaling domain are provided herein below and in the Examples and Figures. The stimulatory signaling activity can be determined; e.g., by enhanced cytokine release, as measured by ELISA (IL-2, IFNλ, TNFα), enhanced proliferative activity (as measured by enhanced cell numbers), or enhanced lytic activity as measured by LDH release assays.

The CAR preferably further comprises at least one co-stimulatory signaling domain which provides additional activity to the reporter CAR-T cell. The CAR may comprise a co-stimulatory signaling domain which is a fragment/polypeptide part of murine/mouse or human CD28 (the UniProt Entry of the human CD28 is P10747 (version number 173 with sequence number 1); the UniProt Entry of the murine/mouse CD28 is P31041 (version number 134 with sequence number 2)), CD137 (the UniProt Entry of the human CD137 is Q07011 (version number 145 with sequence number 1); the UniProt Entry of murine/mouse CD137 is P20334 (version number 139 with sequence number 1)), 0X40 (the UniProt Entry of the human OX40 is P23510 (version number 138 with sequence number 1); the UniProt Entry of murine/mouse OX40 is P43488 (version number 119 with sequence number 1)), ICOS (the UniProt Entry of the human ICOS is Q9Y6W8 (version number 126 with sequence number 1)); the UniProt Entry of the murine/mouse ICOS is Q9WV40 (primary citable accession number) or Q9JL17 (secondary citable accession number) with the version number 102 and sequence version 2)), CD27 (the UniProt Entry of the human CD27 is P26842 (version number 160 with sequence number 2); the Uniprot Entry of the murine/mouse CD27 is P41272 (version number 137 with sequence version 1)), 4-1-BB (the UniProt Entry of the murine/mouse 4-1-BB is P20334 (version number 140 with sequence version 1); the UniProt Entry of the human 4-1-BB is Q07011 (version number 146 with sequence version)), DAP10 (the UniProt Entry of the human DAP10 is Q9UBJ5 (version number 25 with sequence number 1); the UniProt entry of the murine/mouse DAP10 is Q9QUJO (primary citable accession number) or Q9R1E7 (secondary citable accession number) with the version number 101 and the sequence number 1)) or DAP12 (the UniProt Entry of the human DAP12 is 043914 (version number 146 and the sequence number 1); the UniProt entry of the murine/mouse DAP12 is 0054885 (primary citable accession number) or Q9R1E7 (secondary citable accession number) with the version number 123 and the sequence number 1). In certain embodiments the CAR may comprise one or more, i.e., 1, 2, 3, 4, 5, 6 or 7 of the herein defined co-stimulatory signaling domains. Accordingly, in the context of the present invention, the CAR may comprise a fragment/polypeptide part of a murine/mouse or preferably of a human CD28 as first co-stimulatory signaling domain and the second co-stimulatory signaling domain is selected from the group consisting of the murine/mouse or preferably of the human CD27, CD28, CD137, OX40, ICOS, DAP10 and DAP12, or fragments thereof. Preferably, the CAR comprises a co-stimulatory signaling domain which is derived from a human origin. Thus, more preferably, the co-stimulatory signaling domain(s) which is (are) comprised in the CAR may comprise or consist of the amino acid sequence as shown in SEQ ID NO:15 (as encoded by the DNA sequence shown in SEQ ID NO:30).

Thus, the co-stimulatory signaling domain which may be optionally comprised in the CAR is a fragment/polypeptide part of the full length CD27, CD28, CD137, OX40, ICOS, DAP10 and DAP12. The amino acid sequence of the murine/mouse full length CD28 is shown herein as SEQ ID NO:70 (murine/mouse as encoded by the DNA sequences shown in SEQ ID NO:69). However, because human sequences are most preferred in the context of the present invention, the co-stimulatory signaling domain which may be optionally comprised in the CAR protein is a fragment/polypeptide part of the human full length CD27, CD28, CD137, OX40, ICOS, DAP10 or DAP12. The amino acid sequence of the human full length CD28 is shown herein as SEQ ID NO:68 (human as encoded by the DNA sequence shown in SEQ ID NO:67).

In one preferred embodiment, the CAR comprises CD28 or a fragment thereof as co-stimulatory signaling domain. The CAR may comprise a fragment of CD28 as co-stimulatory signaling domain, provided that at least one signaling domain of CD28 is comprised. In particular, any part/fragment of CD28 is suitable for the CAR as long as at least one of the signaling motives of CD28 is comprised. For example, the CD28 polypeptide which is comprised in the CAR may comprise or consist of the amino acid sequence shown in SEQ ID NO:15 (as encoded by the DNA sequence shown in SEQ ID NO:30). In the present invention the intracellular domain of CD28, which functions as a co-stimulatory signaling domain, may comprise a sequence derived from the intracellular domain of the CD28 polypeptide having the sequence(s) YMNM (SEQ ID NO:71) and/or PYAP (SEQ ID NO:72). Preferably, the CAR comprises polypeptides which are derived from human origin. For example, the fragment/polypeptide part of the human CD28 which may be comprised in the CAR may comprise or consist of the amino acid sequence shown in SEQ ID NO:15 (as encoded by the DNA sequence shown in SEQ ID NO:30). Accordingly, in one embodiment, the CAR comprises the sequence as shown in SEQ ID NO:15 or a sequence which has up to 1, 2, 3, 4, 5, 6, 7, 8, 9 or 10 substitutions, deletions or insertions in comparison to SEQ ID NO:15 and which is characterized by having a co-stimulatory signaling activity. Specific configurations of CARs comprising a co-stimulatory signaling domain (CSD) are provided herein below and in the Examples and Figures. The co-stimulatory signaling activity can be determined; e.g., by enhanced cytokine release, as measured by ELISA (IL-2, IFNλ, TNFα), enhanced proliferative activity (as measured by enhanced cell numbers), or enhanced lytic activity as measured by LDH release assays.

As mentioned above, in an embodiment of the present invention, the co-stimulatory signaling domain of the CAR may be derived from the human CD28 gene (Uni Prot Entry No: P10747 (accession number with the entry version: 173 and version 1 of the sequence)) and provides CD28 activity, defined as cytokine production, proliferation and lytic activity of the transduced cell described herein, like a transduced T cell. CD28 activity can be measured by release of cytokines by ELISA or flow cytometry of cytokines such as interferon-gamma (IFN-λ) or interleukin 2 (IL-2), proliferation of T cells measured e.g., by ki67-measurement, cell quantification by flow cytometry, or lytic activity as assessed by real time impedence measurement of the target cell (by using e.g., an ICELLligence instrument as described e.g., in Thakur et al., Biosens Bioelectron. 35(1) (2012), 503-506; Krutzik et al., Methods Mol Biol. 699 (2011), 179-202; Ekkens et al., Infect Immun. 75(5) (2007), 2291-2296; Ge et al., Proc Natl Acad Sci U S A. 99(5) (2002), 2983-2988; Düwell et al., Cell Death Differ. 21(12) (2014), 1825-1837, Erratum in: Cell Death Differ. 21(12) (2014), 161). The co-stimulatory signaling domains PYAP and YMNM are beneficial for the function of the CD28 polypeptide and the functional effects enumerated above. The amino acid sequence of the YMNM domain is shown in SEQ ID NO:71; the amino acid sequence of the PYAP domain is shown in SEQ ID NO:72. Accordingly, in the CAR as provided and used herein, the CD28 polypeptide preferably comprises a sequence derived from intracellular domain of a CD28 polypeptide having the sequences YMNM (SEQ ID NO:71) and/or PYAP (SEQ ID NO:72). These signaling motives may, be present at any site within the intracellular domain of the CARs.

The extracellular domain comprising at least one antigen binding moiety capable of specific binding to the target antigen or the modified recognition domain, the anchoring transmembrane domain that does not have a cleavage site for mammalian proteases, the co-stimulatory signaling domain and the stimulatory signaling domain may be comprised in a single-chain multi-functional polypeptide. A single-chain fusion construct e.g., may consist of (a) polypeptide(s) comprising (an) extracellular domain(s) comprising at least one antigen binding moiety, (an) anchoring transmembrane domain(s), (a) co-stimulatory signaling domain(s) and/or (a) stimulatory signaling domain(s). In alternative embodiments, the CAR comprises an antigen binding moiety which is not a single chain fusion construct, i.e., the antigen binding moiety is a Fab or a crossFab fragment. In such embodiments the CAR is not a single chain fusion construct comprising only one polypeptide chain. Preferably such constructs will comprise a single chain heavy chain fusion polypeptide combined with an immunoglobulin light chain, e.g., the heavy chain fusion polypeptide comprises (an) immunoglobulin heavy chain(s), (an) anchoring transmembrane domain(s), (a) co-stimulatory signaling domain(s) and/or (a) stimulatory signaling domain(s) and is combined with (an) immunoglobulin light chain(s). Accordingly, the extracellular domain, the anchoring transmembrane domain, the co-stimulatory signaling domain and the stimulatory signaling domain may be connected by one or more identical or different peptide linker. For example, the linker between the extracellular domain comprising at least one antigen binding moiety capable of specific binding to the recognition domain and the anchoring transmembrane domain may comprise or consist of the amino acid sequence as shown in SEQ ID NO:20. Accordingly, the anchoring transmembrane domain, the co-stimulatory signaling domain and/or the stimulatory domain may be connected to each other by peptide linkers or alternatively, by direct fusion of the domains.

In some embodiments, the antigen binding moiety comprised in the extracellular domain is a single-chain variable fragment (scFv) which is a fusion protein of the variable regions of the heavy (VH) and light chains (VL) of an antibody, connected with a short linker peptide of 10 to about 25 amino acids. The linker is usually rich in glycine for flexibility, as well as serine or threonine for solubility, and can either connect the N-terminus of the VH with the C-terminus of the VL, or vice versa. For example, the linker may have the amino and amino acid sequence as shown in SEQ ID NO:19. A scFv antigen binding moiety retains the specificity of the original antibody, despite removal of the constant regions and the introduction of the linker. scFv antibodies are, e.g., described in Houston, J. S., Methods in Enzymol. 203 (1991) 46-96).

The CAR or parts thereof may comprise a signal peptide. Such a signal peptide will bring the protein to the surface of the T cell membrane. For example, the signal peptide may have the amino and amino acid sequence as shown in SEQ ID NO:73 (as encoded by the DNA sequence shown in SEQ ID NO:74).

The components of the CARs can be fused to each other in a variety of configurations to generate T cell activating CARs. In some embodiments, the CAR comprises an extracellular domain composed of a heavy chain variable domain (VH) and a light chain variable domain (VL) connected to an anchoring transmembrane domain. In some embodiments, the VH domain is fused at the C-terminus to the N-terminus of the VL domain, optionally through a peptide linker. In other embodiments, the CAR further comprises a stimulatory signaling domain and/or a co-stimulatory signaling domain. In a specific such embodiment, the CAR essentially consists of a VH domain and a VL domain, an anchoring transmembrane domain, and optionally a stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the stimulatory signaling domain. Optionally, the CAR further comprises a co-stimulatory signaling domain. In one such specific embodiment, the antigen binding receptor essentially consists of a VH domain and a VL domain, an anchoring transmembrane domain, a stimulatory signaling domain and a co-stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the stimulatory signaling domain, wherein the stimulatory signaling domain is fused at the C-terminus to the N-terminus of the co-stimulatory signaling domain. In an alternative embodiment, the co-stimulatory signaling domain is connected to the anchoring transmembrane domain instead of the stimulatory signaling domain. In a preferred embodiment, the CAR essentially consists of a VH domain and a VL domain, an anchoring transmembrane domain, a co-stimulatory signaling domain and a stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the co-stimulatory signaling domain, wherein the co-stimulatory signaling domain is fused at the C-terminus to the N-terminus of the stimulatory signaling domain.

In preferred embodiments, one of the binding moieties is a Fab fragment or a crossFab fragment. In one preferred embodiment, the antigen binding moiety is fused at the C-terminus of the Fab or crossFab heavy chain to the N-terminus of the anchoring transmembrane domain, optionally through a peptide linker. In an alternative embodiment, the antigen binding moiety is fused at the C-terminus of the Fab or crossFab light chain to the N-terminus of the anchoring transmembrane domain, optionally through a peptide linker. In other embodiments, the CAR further comprises a stimulatory signaling domain and/or a co-stimulatory signaling domain. In a specific such embodiment, the CAR essentially consists of a Fab or crossFab fragment, an anchoring transmembrane domain, and optionally a stimulatory signaling domain connected by one or more peptide linkers, wherein the Fab or crossFab fragment is fused at the C-terminus of the heavy or light chain to the N-terminus of the anchoring transmembrane domain, wherein the anchoring transmembrane domain is fused at the C-terminus to the N-terminus of the stimulatory signaling domain. Preferably, the CAR further comprises a co-stimulatory signaling domain. In one such embodiment, the CAR essentially consists of a Fab or crossFab fragment, an anchoring transmembrane domain, a stimulatory signaling domain and a co-stimulatory signaling domain connected by one or more peptide linkers, wherein the Fab or crossFab fragment is fused at the C-terminus of the heavy or light chain to the N-terminus of the anchoring transmembrane domain, wherein the stimulatory signaling domain is fused at the C-terminus to the N-terminus of the co-stimulatory signaling domain. In a preferred embodiment, the co-stimulatory signaling domain is connected to the anchoring transmembrane domain instead of the stimulatory signaling domain. In a most preferred embodiment, the CAR essentially consists of a Fab or crossFab fragment, an anchoring transmembrane domain, a co-stimulatory signaling domain and a stimulatory signaling domain, wherein the Fab or crossFab fragment is fused at the C-terminus of the heavy chain to the N-terminus of the anchoring transmembrane domain through a peptide linker, wherein the anchoring transmembrane domain is fused at the C-terminus to the N-terminus of the co-stimulatory signaling domain, wherein the co-stimulatory signaling domain is fused at the C-terminus to N-terminus of the stimulatory signaling domain.

The antigen binding moiety, the anchoring transmembrane domain and the stimulatory signaling and/or co-stimulatory signaling domains may be fused to each other directly or through one or more peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers, wherein “n” is generally a number between 1 and 10, typically between 2 and 4. A preferred peptide linker for connecting the antigen binding moiety and the anchoring transmembrane moiety is GGGGS (G₄S) according to SEQ ID NO 20. An exemplary peptide linker suitable for connecting variable heavy chain (VH) and the variable light chain (VL) is GGGSGGGSGGGSGGGS (G45)4 according to SEQ ID NO 19.

Additionally, linkers may comprise (a portion of) an immunoglobulin hinge region. Particularly where an antigen binding moiety is fused to the N-terminus of an anchoring transmembrane domain, it may be fused via an immunoglobulin hinge region or a portion thereof, with or without an additional peptide linker.

As described herein, the CARs provided and used according to the present invention comprise an extracellular domain comprising at least one antigen binding moiety. A CAR with a single antigen binding moiety is useful and preferred, particularly in cases where high expression of the CAR is needed. In such cases, the presence of more than one antigen binding moiety may limit the expression efficiency of the CAR. In other cases, however, it will be advantageous to have a CAR comprising two or more antigen binding moieties, for example to optimize targeting to the target site or to allow crosslinking of target cell antigens.

In the context of the methods according to the invention, contacting the reporter CAR-T cell (e.g., the Jurkat cell) with a target cell (e.g., the tumor cell) comprising the target antigen on the surface leads to expression of the reporter gene as described herein. Accordingly, in one embodiment, activation of the intracellular signaling and/or co-signaling domain as described herein leads to activation of a response element as herein described. In a preferred embodiment, the response element controls the expression of the reporter gene. In a preferred embodiment, activation of the response element leads to expression of the reporter gene. Accordingly, the reporter gene in the reporter cells is expressed upon binding of the target antigen binding moiety to the target. In one embodiment, the expression of the reporter gene is indicative for binding of the target antigen binding moiety to the target antigen. In this context, the binding of the CAR to its target elicits a cellular response which results in a modulation of the activity of the response element, either directly or through a cascade of cell signaling. The response element is a DNA element which can be silenced or activated by transcription factors or the like.

Response elements are known in the art and are commercially available, e.g., in reporter vectors. Usually the response element comprises DNA repeat elements and is a cis-acting enhancer element located upstream of a minimal promotor which drives expression of a reporter gene upon transcription factor binding.

Binding of the CAR to the target antigen activates the response element. In one embodiment the response element is a nuclear response element located in the nucleus of the cell. In another embodiment said response element is located on a plasmid in the reporter cell. In one embodiment the assay comprises the preliminary step of transfection of the reporter cells, e.g., a Jurkat cell, with an expression vector comprising the DNA sequence coding for the reporter gene under the control of the response element. Additionally, the reporter cells can be transfected with an expression vector comprising the DNA sequence coding for the CAR. The reporter cells can be transfected with an expression vector comprising all elements of the signaling cascade or with different vectors individually expressing the different components. In one embodiment, the reporter cells comprise the DNA sequence coding for the reporter gene under the control of the response element, and the DNA sequence coding for the CAR.

Accordingly, as described herein, the CAR is functionally linked to a response element. In one embodiment, the response element controls the expression of the reporter gene. In one embodiment the response element is part of the NFAT pathway, the NF-κB pathway or the AP-1 pathway, preferably, the NFAT pathway.

In one embodiment the reporter gene is selected from a gene coding for a fluorescent protein or a gene coding for an enzyme whose catalytic activity can be detected. In one embodiment, the reporter gene is coding for a luminescent protein. In further embodiments the fluorescent protein is selected from the group consisting of green fluorescent protein (GFP), yellow fluorescent protein (YFP), red fluorescent protein (RFP), Blue fluorescent protein (BFP, Heim et al. 1994, 1996), a cyan fluorescent variant known as CFP (Heim et al. 1996; Tsien 1998); a yellow fluorescent variant known as YFP (Oruro et al. 1996; Wachter et al. 1998); a violet-excitable green fluorescent variant known as Sapphire (Tsien 1998; Zapata-Hommer et al. 2003); and a cyan-excitable green fluorescing variant known as enhanced green fluorescent protein or EGFP (Yang et al. 1996) enhanced green fluorescent protein (EGFP) and can be measured e.g., by live cell imaging (e.g., Incucyte) or fluorescent spectrophotometry. In one embodiment the enzyme whose catalytic activity can be detected is selected from the group consisting of luciferase, beta Galactosidase and Alkaline Phosphatase. In one embodiment the reporter gene is encoding for GFP. In a preferred embodiment the reporter gene is encoding for luciferase. The activity of luciferase can be detected by commercially available assays, e.g., by the Luciferase 1000 Assay System or the ONE-Glo™ Luciferase Assay System (both Promega). The Luciferase 1000 Assay System contains coenzyme A (CoA) besides luciferin as a substrate, resulting in a strong light intensity lasting for at least one minute. For assaying the intracellular luciferase, it is necessary to lyse the cells prior to detection. The light which is produced as a by-product of the reaction is collected by the luminometer from the entire visible spectrum. In the examples shown herein the signal was proportional to the amount of produced luciferase and therefore proportional to the strength of the activation of the NFAT promotor. In another embodiment a Luciferase assay is used wherein the luciferase is secreted from the cells. Hence the assay can be performed without lysis of the cells.

As described herein, the expression of the reporter gene can be directly correlated with the binding of the target antigen binding moiety to the target cell and the resulting activation of the reporter CAR-T cell, e.g., the transduced Jurkat cell. For example when using a gene encoding for luciferase as a reporter gene, the amount of light detected from the cells correlates directly with the target antigen binding and is indicative for the target antigen binding when compared to appropriate control situations.

In one embodiment, the target antigen is a cell surface antigen and/or receptor. In one embodiment, the target antigen is selected from the group consisting of CD20, CD38, CD138, CEA, EGFR, Fo1R1, HER2, LeY, MCSP, STEAP1, TYRP1, and WT1, or a fragment thereof. However, the target antigen is not limited to proteins located on the cell surface but may also derive from polypeptides or proteins which are temporarily or permanently located intracellularly. In such cases, the target antigen deriving from an intracellular polypeptide or protein can be presented on the cell surface by one or several molecules of the maj or histocompatibility complex (MHC). Indeed, currently available diagnostic assays are usually limited to proteins presented on the cell surface, however, peptides presented by MHC complex remain challenging targets. It is a particular advantage of the diagnostic assays of the present invention that peptide/MHC complexes can be specifically detected with high sensitivity. Accordingly, in one embodiment, the target antigen is a peptide bound to a molecule of the MHC. In one embodiment, the MHC is a human MHC. In one embodiment, the peptide bound to a molecule of the MHC has an overall length of between 8 and 100, preferably between 8 and 30, and more preferred between 8 and 16 amino acids. In one embodiment, the target antigen derives from a protein which is exclusively or mainly expressed in tumor tissue. In one embodiment, the protein is an intracellular protein and the peptide is generated by the MHC-I or MHC-II pathway and presented by a MHC class I or MHC class II complex. In one embodiment, the peptide is generated by the MHC-I pathway and presented by a MHC class I complex. In one embodiment, the target antigen binding moiety is a T cell receptor like (TCRL) antigen binding moiety. A TCRL antigen binding moiety is capable of specific binding to a peptide antigen which is exclusively or mainly expressed in tumor tissue, wherein the peptide antigen is bound to a molecule of the MHC located on the surface of a target cell, particularly a cancer cell. In this context, the methods of the present invention are suitable to detect the presence of a target cell, e.g., a tumor cell, based on presence of a specific peptide/MHC complex on the surface of the target cell using established or novel TCRL target antigen binding moieties.

The binding of the CAR to the target antigen can be determined qualitatively or qualitatively, i.e., by the presence or absence of the expression of the reporter gene; with the absence of any fluorescence or luminescence being indicative of no binding. For quantitative measurement of binding and activation the amount of reporter gene activation can be compared to a reference. Accordingly, the diagnostic assay as described herein may additionally comprise the step of comparing the level of expression of the reporter gene to a reference. A suitable reference usually comprises a negative control which is substantially identical to the referenced assay omitting one or several essential component(s) of the assay or method. For the methods of the invention the omitted component may be, e.g., omitting the target cell inclusion of a cell not expressing the target antigen. Alternatively, a reporter CAR-T cell (e.g., a Jurkat cell) not capable of binding to the target cell and/or the recognition domain of the antigen binding molecule can be used. In a one embodiment, the reference is expression of the reporter gene in absence of the target cell. In another embodiment, the reference is expression of the reporter gene in presence of the Jurkat cell not expressing the CAR capable of specific binding to the target cell. In specific embodiments, the expression of the reporter gene is at least 2×, 3×, 4×, 5×, 10×, 100×, 1000×, or 10000×, higher compared to the expression of the reporter gene in presence of the reference.

Alternatively, the absence of reporter gene expression can be defined by a certain threshold, i.e., after deduction of a background signal. The background signal is usually determined by performing the assay with all reagents but in absence of the target antigen. A positive signal from the diagnostic assay according to the invention is given if the level of expression of the reporter gene in the presence of the target antigen in relation to the expression of the reporter gene in absence of the target antigen is higher than a predefined threshold value.

In specific embodiments, the threshold value is 2, 3, 4, 5, 10, 100, 1000, or 10000.

The novel diagnostic assay as described herein is robust, suitable for use in high-throughput format and efficient in terms of hands-on time needed to accomplish the assay. Furthermore, the diagnostic assay of the present invention tolerates the presence of dead cells in the sample to be analyzed. This is in contrast to cell assays wherein the binding and functionality of an antigen binding molecule is determined by measuring cell viability or cell death.

One further advantage of the new diagnostic assay described herein is that no washing steps are required. The reporter cells and/or the antigen binding molecule to be tested can be added to the target cells, e.g., tumor cells, in either order or at the same time. In one embodiment, reporter CAR-T cells and the tumor sample is added to cell culture medium in a suitable cell culture format, e.g., in a well of a 24 well plate or in a well of a 96 well plate. Preferably the testing medium is a medium that provides conditions for cells to be viable for up to 48 hours. In one embodiment the diagnostic assay is performed in a microtiter plate. In one embodiment the microtiter plate is suitable for high throughput screening. The diagnostic assay of the present invention can be performed in any format that allows for rapid preparation, processing, and analysis of multiple reactions. This can be, for example, in multi-well assay plates (e.g., 24 wells, 96 wells or 384 wells). Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting fluorescent and/or luminescent signals.

In one embodiment about 100000 to about 1000000 reporter CAR-T cells (e.g., Jurkat cells) per well of a 24-well plate are provided in step c). In a preferred embodiment about 300000 to about 700000 cells or about 400000 to about 600000 reporter CAR-T cells (e.g., Jurkat cells) per well of a 24-well plate are provided in step c). In one embodiment about 500000 reporter CAR-T cells (e.g., Jurkat cells) per well of a 24-well plate are provided in step c). In one embodiment about 10000 to about 100000 reporter CAR-T (e.g., Jurkat cells) per well of a 96-well plate are provided in step c). In a preferred embodiment about 30000 to about 70000 reporter CAR-T cells or about 40000 to about 60000 reporter CAR-T cells (e.g., Jurkat cells) per well of a 96-well plate are provided in step c). In one embodiment about 50000 reporter CAR-T cells (e.g., Jurkat cells) per well of a 96-well plate are provided in step c). In one embodiment about 3000 to about 30000 reporter CAR-T cells (e.g., Jurkat cells) per well of a 384-well plate are provided in step c). In a preferred embodiment about 5000 to about 15000 cells or about 8000 to about 12000 reporter CAR-T cells (e.g., Jurkat cells) per well of a 384-well plate are provided in step c). In one embodiment about 10000 reporter CAR-T cells (e.g., Jurkat cells) per well of a 384-well plate are provided in step c). In one embodiment about 200000 to about 2000000 reporter CAR-T (e.g., Jurkat cells) per ml of cell culture medium are provided in step c). In a preferred embodiment about 600000 to about 1400000 reporter CAR-T (e.g., Jurkat cells) or about 800000 to about 1200000 reporter CAR-T (e.g., Jurkat cells) per ml of cell culture medium are provided in step c). In one embodiment about 1000000 reporter CAR-T (e.g., Jurkat cells) per ml of cell culture medium are provided in step c).

Furthermore provided are transduced T cells, i.e., reporter CAR-T cells, (e.g., transduced Jurkat cells), capable of expressing a CAR as described herein and their use in the diagnostic assay according to the invention. The CAR relates to a molecule which is naturally not comprised in and/or on the surface of T cells and which is not (endogenously) expressed in or on normal (non-transduced) T cells. Thus, the CAR as used herein in and/or on T cells is artificially introduced into T cells. The CAR molecule, artificially introduced and subsequently presented in and/or on the surface of said T cells, e.g., reporter CAR-T cells, comprises domains comprising one or more antigen binding moiety accessible (in vitro or in vivo) to antigens. In this context, these artificially introduced molecules are presented in and/or on the surface of said T cells after transduction as described herein below. Accordingly, after transduction, T cells according to the disclosure can be activated by the target antigen.

Herein provided are also transduced T cells (e.g., Jurkat cells) expressing a CAR encoded by (a) nucleic acid molecule(s) encoding the CAR as described herein. Accordingly, in the context of the present invention, the transduced cell may comprise a nucleic acid molecule encoding the CAR as provided and used herein.

In the context of the present invention, the term “transduced T cell” (e.g., transduced Jurkat cells) relates to a genetically modified T cell (i.e., a T cell wherein a nucleic acid molecule has been introduced deliberately). In particular, the nucleic acid molecule encoding the CAR as described herein can be stably integrated into the genome of the T cell by using a retroviral or lentiviral transduction. The extracellular domain of the CAR may comprise the complete extracellular domain of an antigen binding moiety as described herein but also parts thereof. The minimal size required being the antigen binding site of the antigen binding moiety in the CAR. The extracellular portion of the CAR (i.e., the extracellular domain comprising the antigen binding moiety) can be detected on the cell surface, while the intracellular portion (i.e., the co-stimulatory signaling domain(s) and the stimulatory signaling domain) are not detectable on the cell surface. The detection of the extracellular domain of the CAR can be carried out by using an antibody which specifically binds to this extracellular domain or by the target antigen, which the extracellular domain is capable to bind. The extracellular domain can be detected using these antibodies or antigens by flow cytometry or microscopy.

The transduced cells may be any immune cell. These include but are not limited to B-cells, T cells, Natural Killer (NK) cells, Natural Killer (NK) T cells, λδ T cells, innate lymphoid cells, macrophages, monocytes, dendritic cells, or neutrophils and immortalized cell lines thereof (e.g., Jurkat cells). Preferentially, said immune cell would be a lymphocyte, preferentially a NK or T cells. The said T cells include CD4 T cells and CD8 T cells. Triggering of the CAR on the surface of the leukocyte will render the cell responsive against a target cell irrespective of the lineage the cell originated from. Activation will happen irrespective of the stimulatory signaling domain or co-stimulatory signaling domain chosen for the CAR and is not dependent on the exogenous supply of additional cytokines.

The transduced cell may be co-transduced with further nucleic acid molecules, e.g., with a nucleic acid molecule encoding a response element as described herein.

The transduced cell/cells is/are preferably grown under controlled conditions, outside of their natural environment. In particular, the term “culturing” means that cells (e.g., the transduced cell(s)) are in vitro. Culturing cells is a laboratory technique of keeping cells alive which are separated from their original tissue source. Herein, the transduced cell used according to the present invention is cultured under conditions allowing the expression of the introduced gene in or on said transduced cells. Conditions which allow the expression of a transgene are commonly known in the art.

Further provided herein are nucleic acids and vectors encoding one or several CARs used according to the present invention. The nucleic acid molecules may be under the control of regulatory sequences. For example, promoters, transcriptional enhancers and/or sequences which allow for induced expression of the CARs may be employed. In the context of the present invention, the nucleic acid molecules are expressed under the control of constitutive or inducible promoter. Suitable promoters are e.g., the CMV promoter (Qin et al., PLoS One 5(5) (2010), e10611), the UBC promoter (Qin et al., PLoS One 5(5) (2010), e10611), PGK (Qin et al., PLoS One 5(5) (2010), e10611), the EF1A promoter (Qin et al., PLoS One 5(5) (2010), e10611), the CAGG promoter (Qin et al., PLoS One 5(5) (2010), e10611), the SV40 promoter (Qin et al., PLoS One 5(5) (2010), e10611), the COPIA promoter (Qin et al., PLoS One 5(5) (2010), e10611), the ACTSC promoter (Qin et al., PLoS One 5(5) (2010), e10611), the TRE promoter (Qin et al., PLoS One. 5(5) (2010), e10611), the Oct3/4 promoter (Chang et al., Molecular Therapy 9 (2004), S367-S367 (doi: 10.1016/j.ymthe.2004.06.904)), or the Nanog promoter (Wu et al., Cell Res. 15(5) (2005), 317-24). Herein the term vector relates to a circular or linear nucleic acid molecule which can autonomously replicate in a cell (i.e., in a transduced cell) into which it has been introduced. Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook et al. (loc cit.) and Ausubel, Current Protocols in Molecular Biology, Green Publishing Associates and Wiley Interscience, N.Y. (1989), (1994). Alternatively, the polynucleotides and vectors can be reconstituted into liposomes for delivery to target cells. Relevant sequences can be transferred into expression vectors where expression of a particular polypeptide is required. Typical cloning vectors include pBluescript SK, pGEM, pUC9, pBR322, pGA18 and pGBT9. Typical expression vectors include pTRE, pCAL-n-EK, pESP-1, pOP13CAT. The vector can be polycistronic. Such regulatory sequences (control elements) are known to the skilled person and may include a promoter, a splice cassette, translation initiation codon, and translation and insertion site for introducing an insert into the vector(s). In the context of the present invention, said nucleic acid molecule(s) is (are) operatively linked to said expression control sequences allowing expression in eukaryotic or prokaryotic cells. It is envisaged that said vector(s) is (are) an expression vector(s) comprising the nucleic acid molecule(s) encoding the CAR as defined herein. Operably linked refers to a juxtaposition wherein the components so described are in a relationship permitting them to function in their intended manner. A control sequence operably linked to a coding sequence is ligated in such a way that expression of the coding sequence is achieved under conditions compatible with the control sequences. In case the control sequence is a promoter, it is obvious for a skilled person that double-stranded nucleic acid is preferably used.

In the context of the present invention the recited vector(s) is (are) an expression vector(s). An expression vector is a construct that can be used to transform a selected cell and provides for expression of a coding sequence in the selected cell. An expression vector(s) can for instance be cloning (a) vector(s), (a) binary vector(s) or (a) integrating vector(s). Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40 , RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences encoding signal peptides capable of directing the polypeptide to a cellular compartment or secreting it into the medium may be added to the coding sequence of the recited nucleic acid sequence and are well known in the art; see also, e.g., appended Examples.

The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a CAR including an N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogene), pEF-DHFR, pEF-ADA or pEF-neo (Raum et al. Cancer Immunol Immunother 50 (2001), 141-150) or pSPORT1 (GIBCO BRL).

The described nucleic acid molecule(s) or vector(s) which is (are) introduced in the T cell or its precursor cell may either integrate into the genome of the cell or it may be maintained extrachromosomally.

### Exemplary Embodiments

1. A diagnostic assay for determining the presence of a tumor cell in a sample, the diagnostic assay comprising the steps of:
   a) contacting the sample with a chimeric antigen receptor (CAR) expressing reporter T (CAR-T) cell wherein the reporter CAR-T cell comprises:
      i. a CAR capable of specific binding to the the tumor cell, wherein the CAR is operationally coupled to a response element;
      ii. a reporter gene under the control of the response element; and
   b) determining T cell activation by measuring the expression of the reporter gene to establish the presence of the tumor cell.
2. The diagnostic assay of embodiment 1 or 2, wherein the CAR comprises a target antigen binding moiety capable of specific binding to a tumor target antigen.
3. The diagnostic assay of embodiment 2, wherein the tumor target antigen is a cell surface antigen and/or receptor.
4. The diagnostic assay of embodiment 2 or 3, wherein the tumor target antigen is selected from the group consisting of CD20, CD38, CD138, CEA, EGFR, Fo1R1, HER2, LeY, MCSP, STEAP1, TYRP1, and WT1, or a fragment thereof.
5. The diagnostic assay of any one of embodiments 2 to 4, wherein the tumor target antigen is a peptide bound to a molecule of the human major histocompatibility complex (MHC).
6. The diagnostic assay of any one of embodiments 2 to 5, wherein the target antigen binding moiety is a T cell receptor like (TCRL) antigen binding moiety.
7. The diagnostic assay of any one of embodiments 2 to 6, wherein the target antigen binding moiety is a Fab fragment.
8. The diagnostic assay of any one of embodiments 1 to 7, wherein the reporter CAR-T cell is a Jurkat cell.
9. The diagnostic assay of any one of embodiments 1 to 8, wherein the CAR comprises at least one intracellular stimulatory signaling and/or co-stimulatory signaling domain.
10. The diagnostic assay of embodiment 9, wherein binding of the target antigen binding moiety to the target antigen leads to activation of the intracellular signaling and/or co-signaling domain.
11. The diagnostic assay of any one of embodiments 9 or 10, wherein activation of the intracellular signaling and/or co-signaling domain leads to activation of the response element.
12. The diagnostic assay of any one of embodiments 1 to 11, wherein the response element controls the expression of the reporter gene.
13. The diagnostic assay of any one of embodiments 1 to 12, wherein activation of the response element leads to expression of the reporter gene.
14. The diagnostic assay of any one of embodiments 1 to 13, wherein the response element is part of the NFAT pathway, the NF-κB pathway or the AP-1 pathway.
15. The diagnostic assay of any one of embodiments 1 to 14, wherein the reporter gene is coding for a luminescent protein.
16. The diagnostic assay of any one of embodiments 1 to 15, wherein the reporter gene is coding for green fluorescent protein (GFP) or luciferase.
17. The diagnostic assay of any one of embodiments 1 to 16, wherein the sample is a patient sample derived from an individual suffering from a disease, in particular wherein the disease is cancer.
18. The diagnostic assay of any one of embodiments 1 to 17, additionally comprising the step of:
   c) comparing the expression of the reporter gene to a reference.
19. The diagnostic assay of embodiment 18, wherein the reference is expression of the reporter gene in the presence of a reference sample, wherein the reference sample does not comprise the tumor cell.
20. The diagnostic assay of embodiment 19, wherein expression of the reporter gene in the presence of the patient sample is at least 2×, 3×, 4×, 5×, 10×, 100×, 1000×, or 10000×, higher compared to the expression of the reporter gene in the presence of the reference sample.
21. The diagnostic assay of any one of embodiments 1 to 20, wherein the sample is a patient sample.
22. The diagnostic assay of embodiment 21, additionally comprising the step of:
   d) establishing the presence of a tumor cell if the expression of the reporter gene in the presence of the patient sample in relation to the expression of the reporter gene in the presence of a reference is higher than a predefined threshold value.
23. The diagnostic assay of embodiment 22, wherein the reference is expression of the reporter gene in the presence of a reference sample, wherein the reference sample does not comprise the tumor cell.
24. The diagnostic assay of embodiment 22 or 23, wherein the threshold value is 2, 3, 4, 5, 10, 100, 1000, or 10000.
25. The diagnostic assay of any one of embodiment 1 to 24, wherein the patient is a mammal, in particular wherein the patient is a human.
26. The diagnostic assay of any one of embodiments 1 to 25 for use in the diagnosis of cancer.
27. A method for monitoring the efficacy of an antitumor treatment, comprising providing a sample from a subject having received antitumor treatment, and determining the presence of tumor cells using the diagnostic assay of any one of embodiments 1 to 25.
28. A method for predicting the efficacy of an antitumor CAR-T cell treatment, comprising providing a sample from a subject having a tumor, and determining T cell activation by measuring the expression of the reporter gene according to the diagnostic assay of any one of embodiments 1 to 25, wherein activation of the reporter gene is indicative for efficacy of the antitumor CAR-T cell treatment when applied to the subject.
29. The diagnostic assays and methods as hereinbefore described with reference to any of the

Examples or to any one of the accompanying drawings.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g., by SDS-PAGE and size exclusion chromatography (SEC).

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2 × PBS on a Dionex HPLC-System. The eluted protein was quantified by UV absorbance and integration of peak areas. BioRad Gel Filtration Standard 151-1901 served as a standard.

Antibody Production

The respective antibodies were produced by co-transfecting HEK293-EBNA cells with the mammalian expression vectors using polyethylenimine. The cells were transfected with the corresponding expression vectors for heavy and light chains in a 1:1 ratio.

Lentiviral Transduction of Jurkat NFAT CAR-T Cells

To produce lentiviral vectors, respective DNA sequences for the correct assembly of the CAR were cloned in frame in a lentiviral polynucleotide vector under a constitutively active human cytomegalovirus immediate early promoter (CMV). The retroviral vector contained a woodchuck hepatitis virus posttranscriptional regulatory element (WPRE), a central polypurine tract (cPPT) element, a pUC origin of replication and a gene encoding for antibiotic resistance facilitating the propagation and selection in bacteria.

To produce functional virus particles, Lipofectamine LTX™ based transfection was performed using 60-70% confluent Hek293T cells (ATCC CRL3216) and CAR containing vectors as well as pCMV-VSV-G:pRSV-REV:pCgpV transfer vectors at 3:1:1:1 ratio. After 48h supernatant was collected, centrifuged for 5 minutes at 250 g to remove cell debris and filtrated through 0.45 μm or 0.22 μm polyethersulfon filters. Concentrated virus particles (Lenti-x-Concentrator, Takara) were used to transduce Jurkat NFAT cells (Signosis). Positive transduced cells were sorted as pool or single clones using a FACS-ARIA sorter (BD Bioscience). After cell expansion to appropriate density Jurkat NFAT reporter CAR-T cells were used for experiments.

### Example 1

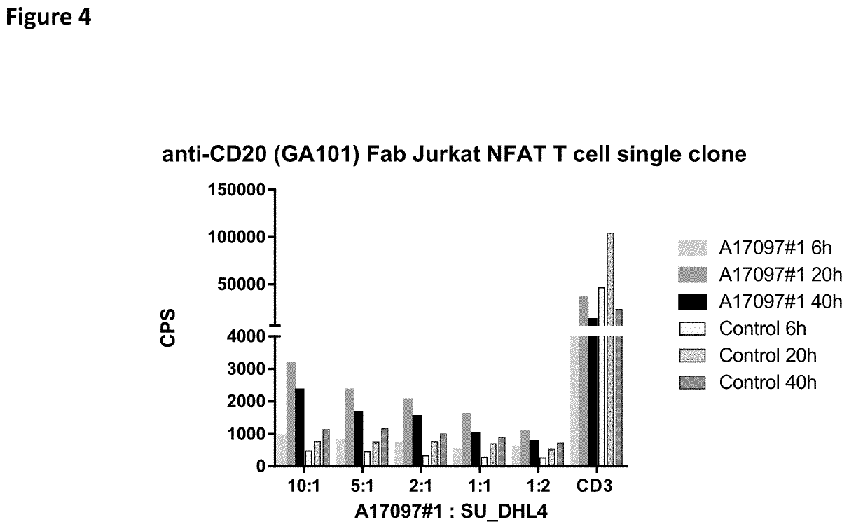

Described herein is a Jurkat NFAT T cell reporter assay using CD20 expressing SUDHDL4 tumor cells as target cells and a sorted single clone of anti-CD20-Fab-CD28ATD-CD28CSD-CD3zSSD expressing Jurkat NFAT T cells as target cells (FIG. 4). As positive control, some wells of a 96 well plate (Cellstar Greiner-bio-one, CAT-No. 655185) were coated with 10 μg/ml CD3 antibody (from Biolegend®) in phosphate buffered saline (PBS) either for 4° C. over night or for at least lh at 37° C. The CD3 antibody coated wells were washed twice with PBS, after the final washing step PBS was fully removed. Jurkat NFAT wild type cells or Jurkat NFAT CAR cells engineered to express the antigen binding receptor anti-CD20-Fab-CD28ATD-CD28CSD-CD3zSSD (further termed as reporter cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells expressing the antigen of interest, were counted and checked for their viability as well. Cell number was adjusted to 1×10⁶ viable cells/ml in growth medium. Target cells and reporter cells were plated in 10:1, 5:1, 2:1 or 1:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using a Tecan® Spark10M plate reader, 1 sec/well as detection time.

The bar diagram shows the activation of anti-CD20-Fab-CD28ATD-CD28CSD-CD3zSSD expressing Jurkat NFAT T cells dependent on different E:T ratios and dependent of the time of co-cultivation with target cells. It is shown that Jurkat NFAT T cell activation is dependent on the duration of the co-cultivation with target cells and dependent on the E:T ratio. For all tested conditions an incubation time of 20 hours displays the highest luminescence signal. Further, among the different E:T ratios the 10:1 E:T ratio depicts the highest detectable luminescence signal. Jurkat NFAT wild type T cells show only a time dependent increase in luminescence signal, whereby after 40 hours the highest luminescence signal can be detected. The detected luminescence signal is independent of E:T ratio and in general also clearly lower than each luminescence signal detected for anti-CD20-Fab-CD28ATD-CD28CSD-CD3zSSD expressing Jurkat NFAT T cells at the respective time points. In general, the highest luminescence signal is detectable if cells were incubated in CD3 antibody coated wells. The anti-CD20-Fab-CD28ATD-CD28CSD-CD3zSSD expressing Jurkat NFAT T cells depict a higher signal compared to not transduced Jurkat NFAT control T cells. Each point represents the mean of a technical duplicate.

### Example 2

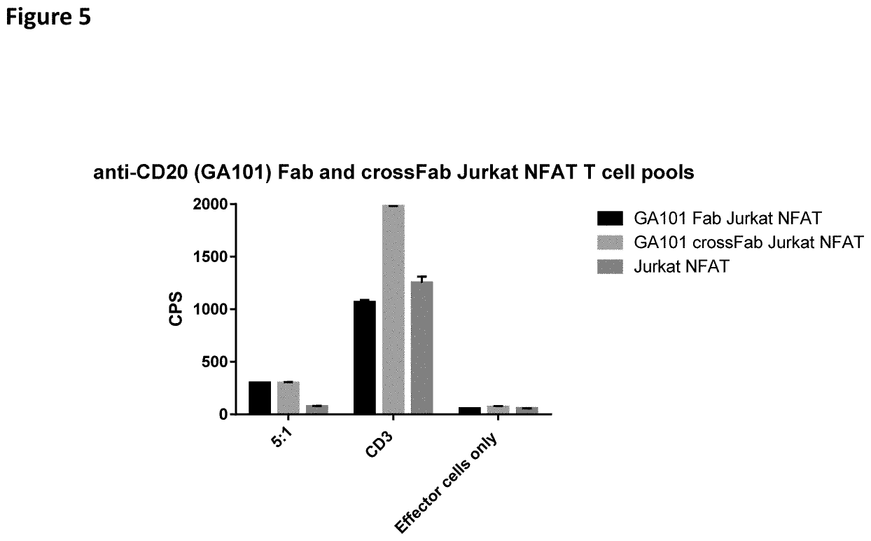

Described herein is a Jurkat NFAT T cell reporter assay using CD20 expressing SUDHDL4 tumor cells as target cells and and a sorted pool of anti-CD20-Fab-CD28ATD-CD28CSD-CD3zSSD or anti-CD20-crossFab-CD28ATD-CD28CSD-CD3zSSD expressing Jurkat NFAT T cells as target cells (FIG. 5). As positive control, wells of a 96 well plate (Cellstar Greiner-bio-one, CAT-No. 655185) were coated with 10 μg/ml CD3 antibody (from Biolegend®) in phosphate buffered saline (PBS) at 4° C. over night. The CD3 antibody coated wells were washed twice with PBS, after the final washing step PBS was fully removed. Jurkat NFAT wild type cells or Jurkat NFAT T cells engineered to express anti-CD20-Fab-CD28ATD-CD28CSD-CD3zSSD or anti-CD20-crossFab-CD28ATD-CD28CSD-CD3zSSD (further termed as reporter cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells, expressing the antigen of interest were counted and checked for their viability as well. Cell number was adjusted to 1×10⁶ viable cells/ml in growth medium. Target cells and reporter cells were plated in 5:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows activation of anti-CD20-Fab-CD28ATD-CD28CSD-CD3zSSD expressing Jurkat NFAT T cells and anti-CD20-crossFab-CD28ATD-CD28CSD-CD3zSSD expressing Jurkat NFAT T cells upon co-cultivation with target cells. If anti-CD20-Fab-CD28ATD-CD28CSD-CD3zSSD or anti-CD20-crossFab-CD28ATD-CD28CSD-CD3zSSD expressing Jurkat NFAT T cells or Jurkat NFAT control T cells were cultivated without target cells, no luminescence signal was detected. The highest luminescence signal was detected when either anti-CD20-Fab-CD28ATD-CD28CSD-CD3zSSD or anti-CD20-crossFab-CD28ATD-CD28CSD-CD3zSSD expressing Jurkat NFAT T cells or Jurkat NFAT control T cells were co-cultivated with target cells in CD3 antibody coated plates. Surprisingly, the crossFab format leads to strong activation of Jurkat NFAT T cells in conjunction with CD3 mediated signaling. Each point represents the mean value of technical triplicates. Standard deviation is indicated by error bars.

### Example 3

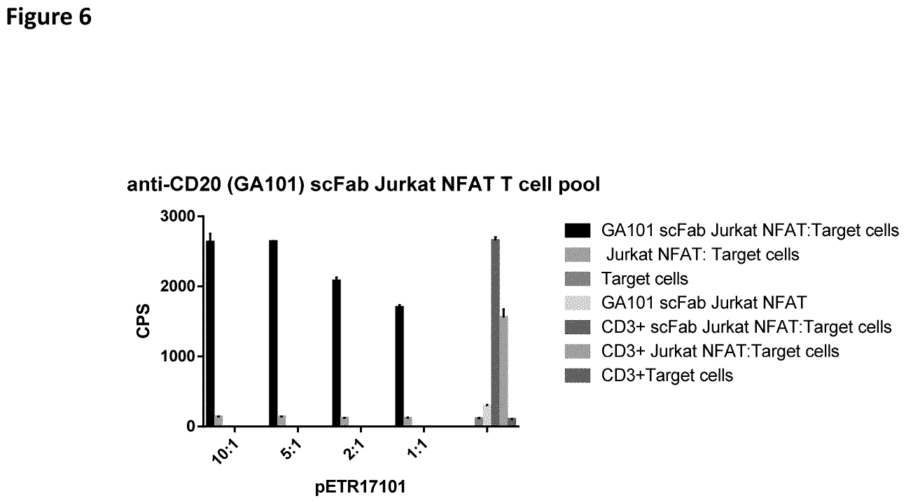

Described herein is a Jurkat NFAT T cell reporter assay performed using CD20 expressing SUDHDL4 tumor cells as target cells and a sorted pool of anti-CD20-scFab-CD28ATD-CD28CSD-CD3zSSD expressing Jurkat NFAT T cells as target cells (FIG. 6).

As positive control, wells of a 96 well plate (Cellstar Greiner-bio-one, CAT-No. 655185) were coated with 10 μg/ml CD3 antibody (from Biolegend®) in phosphate buffered saline (PBS) either for 4° C. over night or for at least lh at 37° C. The CD3 antibody coated wells were washed twice with PBS, after the final washing step PBS was fully removed. Jurkat NFAT wild type T cells or Jurkat NFAT T cells engineered to express anti-CD20-scFab-CD28ATD-CD28CSD-CD3zSSD (further termed as reporter cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells expressing the antigen of interest, were counted and checked for their viability as well. Cell number was adjusted to 1×10⁶ viable cells/ml in growth medium. Target cells and reporter cells were plated in 10:1, 5:1, 2:1 or 1:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®. After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows the activation of anti-CD20-scFab-CD28ATD-CD28CSD-CD3zSSD expressing Jurkat NFAT T cells after 20 hours co-cultivation with SUDHL4 target cells in different E:T ratios. Among the different E:T ratios, the 10:1 and 5:1 E:T ratio show the highest luminescence signal (FIG. 6 black bars). Also anti-CD20-scFab-CD28ATD-CD28CSD-CD3zSSD expressing Jurkat NFAT T cells co-cultured at 10:1 E:T ratio in CD3 antibody coated wells, show a high luminescence signal comparable to the same condition without CD3 stimulus.

Further Jurkat NFAT wild type cells do not show any activation independent of different E:T ratios, but if co-cultivated in 10:1 E:T ratio in CD3 antibody coated wells a clear luminescence signal is delectable, that proves their functionality.

Further control experiments show that target cells or anti-CD20-scFab-CD28ATD-CD28CSD-CD3zSSD expressing T cells alone as well as CD3 antibody coated wells with target cells do not show any activation. Each point represents the mean value of technical triplicates. Standard deviation is indicated by error bars.

### Example 4

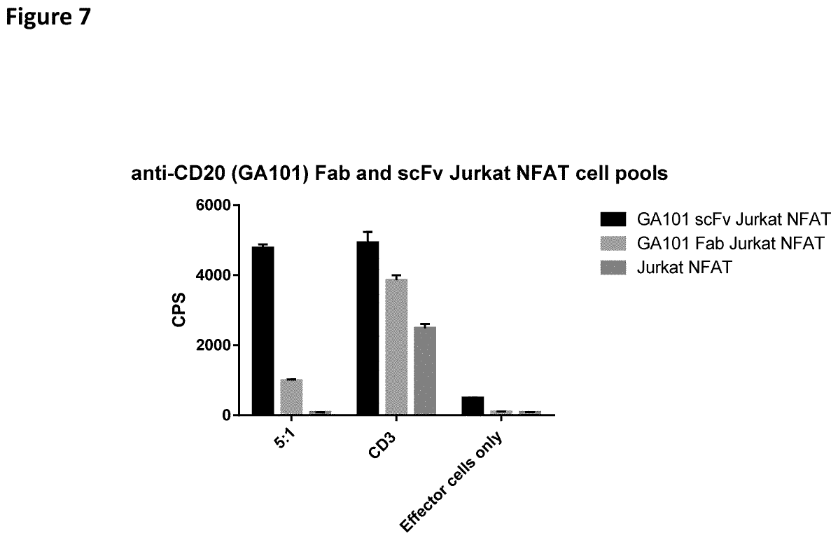

Described herein is a Jurkat NFAT T cell reporter assay performed using CD20 expressing SUDHDL4 tumor cells as target cells and a sorted pool of anti-CD20-Fab-CD28ATD-CD28CSD-CD3zSSD expressing Jurkat NFAT T cells or anti-CD20-scFv-CD28ATD-CD28CSD-CD3zSSD expressing Jurkat NFAT T cells as target cells (FIG. 7).

As positive control, wells of a 96 well plate (Cellstar Greiner-bio-one, CAT-No. 655185) were coated with 10 μg/ml CD3 antibody (from Biolegend®) in phosphate buffered saline (PBS) either for 4° C. over night or for at least 1 h at 37° C. The CD3 antibody coated wells were washed twice with PBS, after the final washing step PBS was fully removed. Jurkat NFAT wild type T cells or Jurkat NFAT T cells engineered to express anti-CD20-Fab-CD28ATD-CD28CSD-CD3zSSD or anti-CD20-scFv-CD28ATD-CD28CSD-CD3zSSD (further termed as reporter cells), were counted and checked for their viability using Cedex HiRes. Cell number was adjusted to 1×10⁶ viable cells/ml. Therefore an appropriate aliquot of the cell suspension was pelleted at 210 g for 5 min at room temperature (RT) and resuspended in fresh RPMI-160+10% FCS+1% Glutamax (growth medium). Target cells expressing the antigen of interest, were counted and checked for their viability as well. Cell number was adjusted to 1×10⁶ viable cells/ml in growth medium. Target cells and reporter cells were plated in 10:1, 5:1, 2:1 or 1:1 E:T ratio (110.000 cells per well in total) in triplicates in a 96-well suspension culture plate (Greiner-bio one) in a final volume of 200 μl. After that the 96 well plate was centrifuged for 2 min at 190 g and RT and sealed with Parafilm®.

After 20 hours at 37° C. and 5% CO₂ in humidity atmosphere incubation the content of each well was mixed by pipetting up and down 10 times using a multichannel pipette. 100 μl cell suspension was transferred to a new white transparent bottom 96 well plate (Greiner-bio-one) and 100 μl ONE-Glo™ Luciferase Assay (Promega) was added. After 15 min incubation in the dark on a rotary shaker at 300 rpm and RT luminescence was measured using Tecan® Spark10M plate reader, 1 sec/well as detection time.

The bar diagram shows the activation of Anti-CD20-scFv-CD28ATD-CD28CSD-CD3zSSD expressing Jurkat NFAT T cells after 20 hours co-cultivation with SUDHL4 target cells at 5:1 E:T ratio. Anti-CD20-scFv-CD28ATD-CD28CSD-CD3zSSD expressing Jurkat NFAT T cells co-cultured with target cells in CD3 antibody coated wells, showed the highest luminescence signal, which is comparable to the same condition without CD3 stimulus. Further Jurkat NFAT wild type cells do not show any activation, but if co-cultivated in 10:1 E:T ratio in CD3 antibody coated wells a clear luminescence signal is delectable, that proves their functionality. Each bar represents the mean value of technical triplicates. Standard deviation is indicated by error bars.

### Example 5

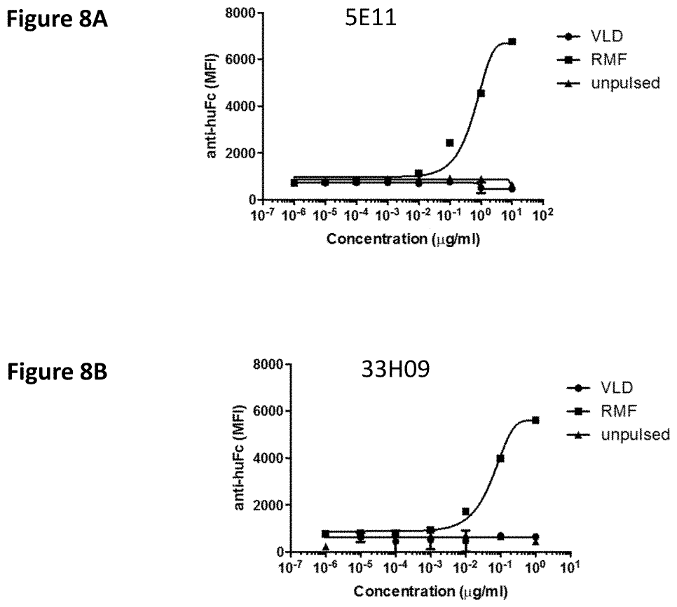

The two antibody candidates 5E11 and 33H09 were originally selected by means of phage display library screening to bind to the WT1-peptide “RMF” in complex with MHCI. Dilution series of both binders in IgG-format were checked for binding by means of flow cytometry. Therefore, T2 cells, pulsed with 10 μM target peptide “RMF”, 10 μM off-target pepide “VLD” or left unpulsed, were incubated with dilution series of the antibodies for 30min on ice. After a washing step removing unbound binders, cells were incubated with fluorescently labelled secondary antibody (anti-huFc, Jackson ImmunoResearch), followed by another washing step and detection of remaining antibodies by flow cytometry. Importantly, in this assay both assessed candidates (5E11 and 33H09) appear similar in terms of specificity, with clear concentration-dependent signal on T2 cells pulsed with target peptide “RMF”, compared to no binding to T2 cells pulsed with off-target pepide “VLD” or unpulsed T2 cells (FIG. 8).

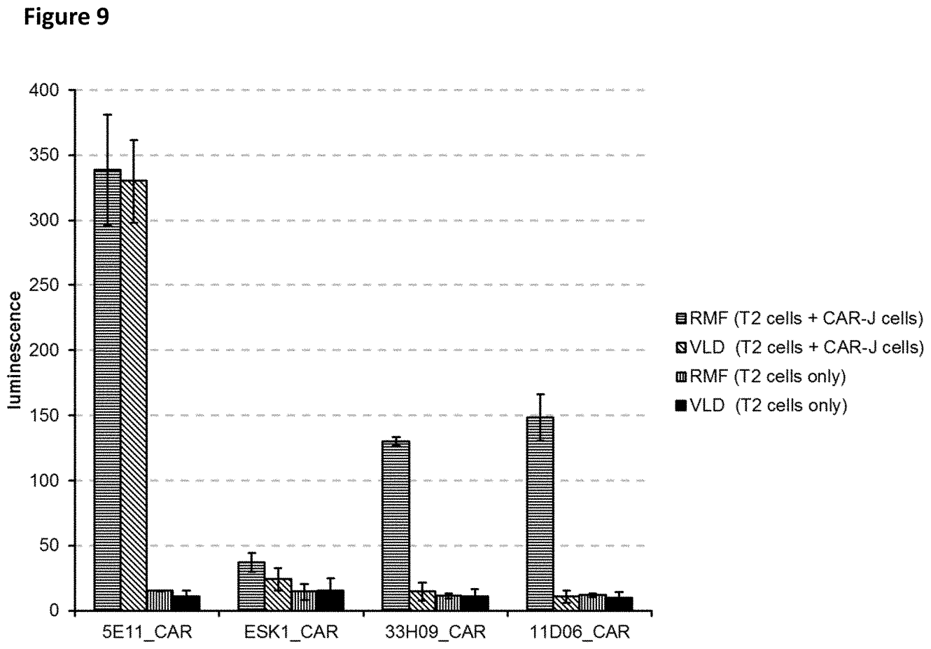

The same two antibody candidates (5E11 and 33H09) plus two further candidates against the same target peptide/MHC (ESK1 and 11D06) were assessed in a Jurkat NFAT reporter CAR-T cell assay depicted in FIG. 9.

This Jurkat NFAT reporter CAR-T cell assay employs pools of Jurkat NFAT reporter cells that recognize HLA-A2/WT1 peptide RMF via four different Fabs (5E11 (SEQ ID NOs 145 and 146), ESK1, 33H09 (SEQ ID NOs 143 and 144) or 11D06 (SEQ ID NOs 141 and 142), respectively), embedded into chimeric antigen receptors expressed on the cell surface.

Prior to co-incubation with the Jurkat NFAT reporter cells, T2 cells were pulsed with the respective peptide at 10⁻⁵M for 2 hours at 37° C., or left unpulsed. Target cells and reporter cells were plated in 5:1 E:T ratio (10.000 effector cells per 2000 target cells per well) in triplicates in a 384-well white flat clear bottom plate (Greiner-bio-one). Jurkat NFAT reporter CAR-T cells and target cells were co-incubated for 7 hours at 37° C., followed by addition of 6 μl per well of ONE-Glo™ luciferase substrate (Promega) and direct measurement of luminescence using a TECAN infinite M1000Pro plate reader. The activation of CAR-NFAT-signaling from triplicate measurements on RMF- or VLD-peptide-pulsed T2 cells is expressed as column graph (FIG. 9). Comparison of signals on RMF-peptide (target) vs. VLD-peptide (off-target) and differently pulsed T2 cells incubated with luciferase substrate, but without reporter cells, helps to assess specificity of activation of the respective binder.

Different from the FACS-based screening depicted in FIG. 8, which could be compared to classical antibody-based diagnostic assays, this Jurkat NFAT reporter CAR-T cell assay does clearly discriminate the different therapeutic candidates 5E11 and 33H09 in terms of specific T-cell activation on target, as opposed to unspecific activation on off-target. Accordingly, the diagnostic assay according to the invention is more suitable to predict therapeutic efficacy of cancer immunotherapies compared to classical antibody-based diagnostic assays.

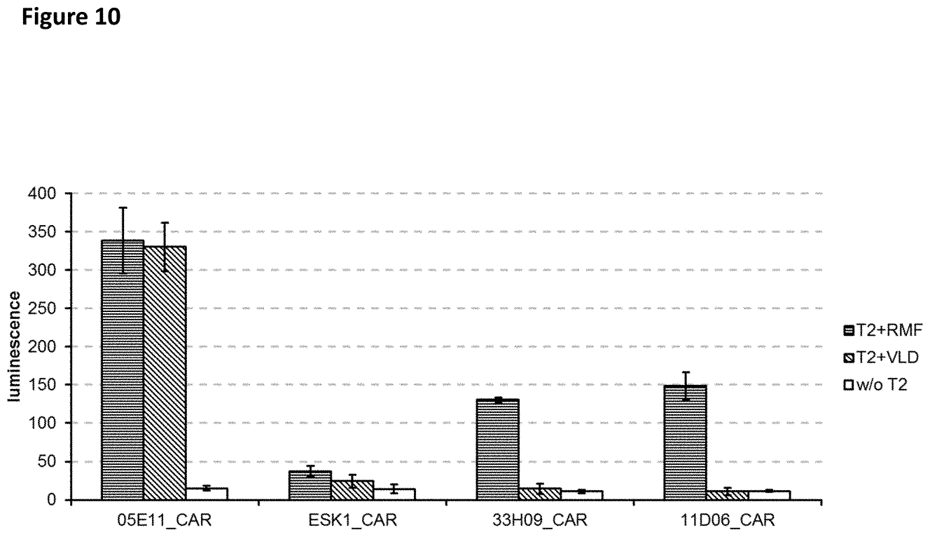

The background signals of the respective Jurkat NFAT reporter cell pools incubated with luciferease substrate as above, but without any co-incubation with target-cell, is low, as depicted in FIG. 10.

### Example 6

This Jurkat NFAT reporter CAR-T cell assay employs pools of Jurkat NFAT reporter cells that recognize two different HLA-A2/peptide targets. Pools F06, F29 and F30 express candidate Fabs that were selected to bind to a blinded peptide/HLA-target, while the pool with Fab 33H09 is specific for HLA-A2/WT1 peptide RMF.

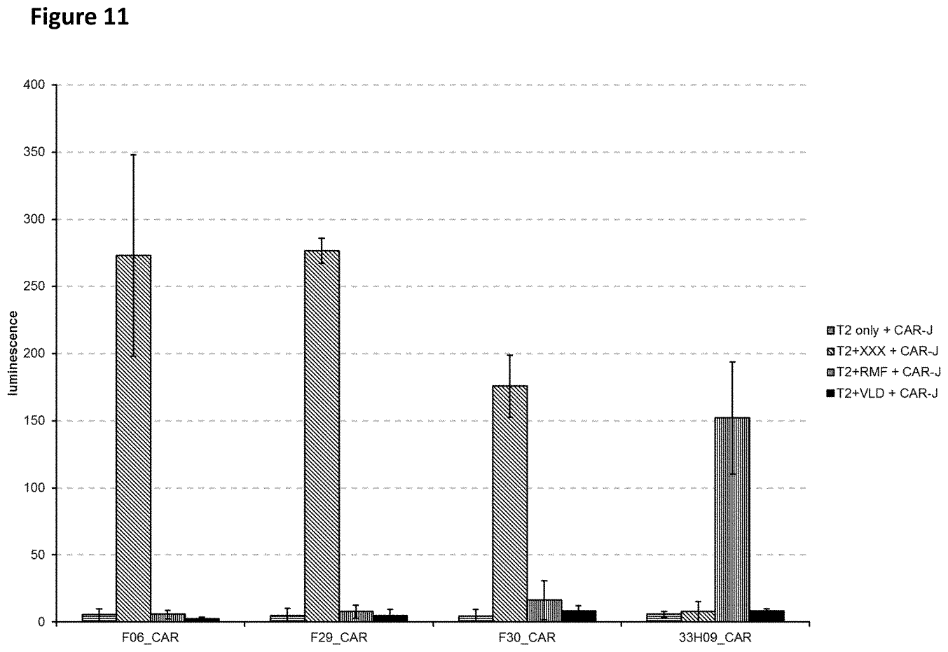

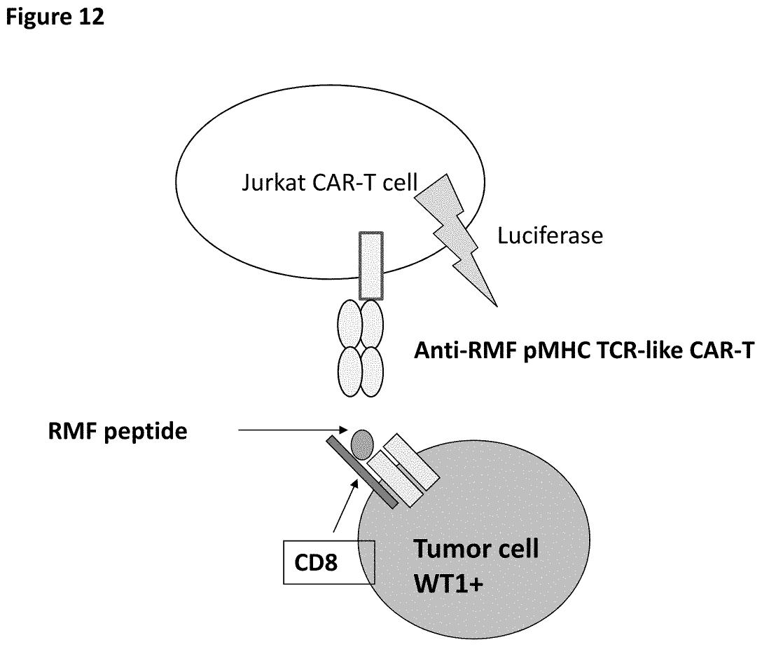

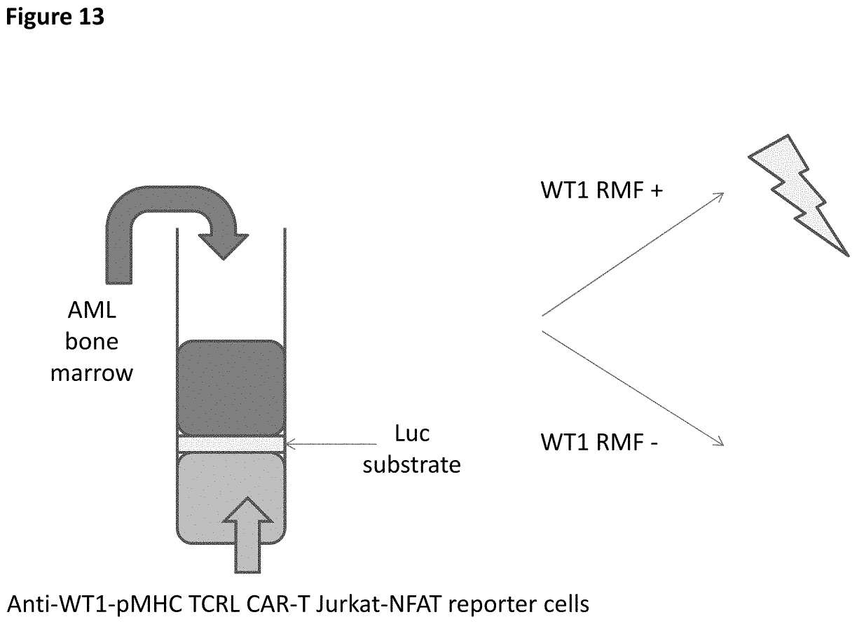

Prior to co-incubation with the Jurkat NFAT reporter cells, T2 cells were pulsed with the respective peptide at 10⁻⁵ M for 2 hours at 37° C., or left unpulsed. Target cells and reporter cells were plated in 5:1 E:T ratio (10.000 effector cells per 2000 target cells per well) in triplicates in a 384-well white flat clear bottom plate (Greiner-bio-one). Jurkat NFAT reporter cells and target cells were co-incubated for 7 hours at 37° C., followed by addition of 6 μl per well of ONE-Glo™ luciferase substrate (Promega) and direct measurement of luminescence using a TECAN infinite M1000Pro plate reader. The activation of CAR-NFAT-signaling from triplicate measurements on T2 cells is expressed as column graph (FIG. 11). Comparison of signals obtained from the four different cell pools on the different peptides indicates the high specificity of activation of the respective candidates towards their desired target peptide/HLA.

Exemplary Sequences

TABLE 2
Anti-CD20 Fab amino acid sequences

| | | SEQ |
|---|---|---|
| | | ID |
| Construct | Protein Sequence | NO |
| Anti-CD20 | YSWIN | 1 |
| CDR H1 Kabat | | |
| Anti-CD20 | RIFPGDGDTDYNGKFKG | 2 |
| CDR H2 Kabat | | |
| Anti-CD20 | NVFDGYWLVY | 3 |
| CDR H3 Kabat | | |
| Anti-CD20 | RSSKSLLHSNGITYLY | 4 |
| CDR L1 Kabat | | |
| Anti-CD20 | QMSNLVS | 5 |
| CDR L2 Kabat | | |
| Anti-CD20 | AQNLELPYT | 6 |
| CDR L3 Kabat | | |
| Anti-CD2O- | QVQLVQSGAEVKKPGSSVKVSCKASGYAFSYSWIN | 7 |
| Fab-heavy | WVRQAPGQGLEWMGRIFPGDGDTDYNGKFKGRVTI | |
| chain- | TADKSTSTAYMELSSLRSEDTAVYYCARNVFDGYW | |
| CD28ATD- | LVYWGQGTLVTVSSASTKGPSVFPLAPSSKSTSGGTA | |
| CD28CSD- | ALGCLVKDYFPEPVTVSWNSGALTSGVHTFPAVLQS | |
| CD3zSSD | SGLYSLSSVVTVPSSSLGTQTYICNVNHKPSNTKVDK | |
| fusion | KVEPKSCGGGGSFWVLVVVGGVLACYSLLVTVAFII | |
| | FWVRSKRSRLLHSDYMNMTPRRPGPTRKHYQPYAPP | |
| | RDFAAYRSRVKFSRSADAPAYQQGQNQLYNELNLG | |
| | RREEYDVLDKRRGRDPEMGGKPRRKNPQEGLYNEL | |
| | QKDKMAEAYSEIGMKGERRRGKGHDGLYQGLSTAT | |
| | KDTYDALHMQALPPR | |
| Anti-CD2O- | QVQLVQSGAEVKKPGSSVKVSCKASGYAFSYSWIN | 8 |
| Fab-heavy | WVRQAPGQGLEWMGRIFPGDGDTDYNGKFKGRVTI | |
| chain | TADKSTSTAYMELSSLRSEDTAVYYCARNVFDGYW | |
| | LVYWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVDK | |
| | KVEPKSC | |
| Anti-CD20- | DIVMTQTPLSLPVTPGEPASISCRSSKSLLHSNGITYLY | 9 |
| Fab-light | WYLQKPGQSPQLLIYQMSNLVSGVPDRFSGSGSGTD | |
| chain | FTLKISRVEAEDVGVYYCAQNLELPYTFGGGTKVEIK | |
| | RTVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |
| Anti-CD20- | DIVMTQTPLSLPVTPGEPASISCRSSKSLLHSNGITYLY | 10 |
| VL | WYLQKPGQSPQLLIYQMSNLVSGVPDRFSGSGSGTD | |
| | FTLKISRVEAEDVGVYYCAQNLELPYTFGGGTKVEIK | |
| CL | RTVAAPSVFIFPPSDEQLKSGTASVVCLLNNFYPREA | 11 |
| | KVQWKVDNALQSGNSQESVTEQDSKDSTYSLSSTLT | |
| | LSKADYEKHKVYACEVTHQGLSSPVTKSFNRGEC | |
| Anti-CD20- | QVQLVQSGAEVKKPGSSVKVSCKASGYAFSYSWIN | 12 |
| VH | WVRQAPGQGLEWMGRIFPGDGDTDYNGKFKGRVTI | |
| | TADKSTSTAYMELSSLRSEDTAVYYCARNVFDGYW | |
| | LVYWGQGTLVTVSS | |
| CH1 | ASTKGPSVFPLAPSSKSTSGGTAALGCLVKDYFPEPV | 13 |
| | TVSWNSGALTSGVHTFPAVLQSSGLYSLSSVVTVPSS | |
| | SLGTQTYICNVNHKPSNTKVDKKVEPKSC | |
| CD28ATD | FWVLVVVGGVLACYSLLVTVAFIIFWV | 14 |
| CD28CSD | RSKRSRLLHSDYMNMTPRRPGPTRKHYQPYAPPRDF | 15 |
| | AAYRS | |
| CD3zSSD | RVKFSRSADAPAYQQGQNQLYNELNLGRREEYDVL | 16 |
| | DKRRGRDPEMGGKPRRKNPQEGLYNELQKDKMAE | |
| | AYSEIGMKGERRRGKGHDGLYQGLSTATKDTYDAL | |
| | HMQALPPR | |
| CD28ATD- | FWVLVVVGGVLACYSLLVTVAFIIFWVRSKRSRLLH | 17 |
| CD28CSD- | SDYMNMTPRRPGPTRKHYQPYAPPRDFAAYRSRVKF | |
| CD3zSSD | SRSADAPAYQQGQNQLYNELNLGRREEYDVLDKRR | |
| | GRDPEMGGKPRRKNPQEGLYNELQKDKMAEAYSEI | |
| | GMKGERRRGKGHDGLYQGLSTATKDTYDALHMQA | |
| | LPPR | |
| eGFP | VSKGEELFTGVVPILVELDGDVNGHKFSVSGEGEGD | 18 |
| | ATYGKLTLKFICTTGKLPVPWPTLVTTLTYGVQCFSR | |
| | YPDHMKQHDFFKSAMPEGYVQERTIFFKDDGNYKT | |
| | RAEVKFEGDTLVNRIELKGIDFKEDGNILGHKLEYNY | |
| | NSHNVYIMADKQKNGIKVNFKIRHNIEDGSVQLADH | |
| | YQQNTPIGDGPVLLPDNHYLSTQSALSKDPNEKRDH | |
| | MVLLEFVTAAGITLGMDELYK | |
| (G4S)4 | GGGGSGGGGSGGGGSGGGGS | 19 |
| linker | | |
| G4S linker | GGGGS | 20 |
| T2A linker | GEGRGSLLTCGDVEENPGP | 21 |

TABLE 3
Anti-CD20 Fab DNA sequences

| | | SEQ |
|---|---|---|
| | | ID |
| Construct | DNA Sequenz | NO |
| Anti-CD20-Fab-CD28ATD-CD28CSD- | ATGGGATGGAGCTGTATCATCCTCTTCTTGGTAGC | 22 |
| CD3zSSD | AACAGCTACCGGTGTGCATTCCGATATCGTGATGA | |
| | CCCAGACTCCACTCTCCCTGCCCGTCACCCCTGGA | |
| | GAGCCCGCCAGCATTAGCTGCAGGTCTAGCAAGA | |
| | GCCTCTTGCACAGCAATGGCATCACTTATTTGTATT | |
| | GGTACCTGCAAAAGCCAGGGCAGTCTCCACAGCTC | |
| | CTGATTTATCAAATGTCCAACCTTGTCTCTGGCGTC | |
| | CCTGACCGGTTCTCCGGATCCGGGTCAGGCACTGA | |
| | TTTCACACTGAAAATCAGCAGGGTGGAGGCTGAG | |
| | GATGTTGGAGTTTATTACTGCGCTCAGAATCTAGA | |
| | ACTTCCTTACACCTTCGGCGGAGGGACCAAGGTGG | |
| | AGATCAAACCGTACGGTGGCTGCACCATCTGTCTT | |
| | CATCTTCCCGCCATCTGATGAGCAGTTGAAATCTG | |
| | GAACTGCCTCTGTTGTGTGCCTGCTGAATAACTTCT | |
| | ATCCCAGAGAGGCCAAAGTACAGTGGAAGGTGGA | |
| | TAACGCCCTCCAATCGGGTAACTCCCAGGAGAGTG | |
| | TCACAGAGCAGGACAGCAAGGACAGCACCTACAG | |
| | CCTCAGCAGCACCCTGACGCTGAGCAAAGCAGACT | |
| | ACGAGAAACACAAAGTCTACGCCTGCGAAGTCAC | |
| | CCATCAGGGCCTGAGCTCGCCCGTCACAAAGAGCT | |
| | TCAACAGGGGAGAGTGTTAGAATAGAATTCCCCG | |
| | AAGTAACTTAGAAGCTGTAAATCAACGATCAATAG | |
| | CAGGTGTGGCACACCAGTCATACCTTGATCAAGCA | |
| | CTTCTGTTTCCCCGGACTGAGTATCAATAGGCTGCT | |
| | CGCGCGGCTGAAGGAGAAAACGTTCGTTACCCGA | |
| | CCAACTACTTCGAGAAGCTTAGTACCACCATGAAC | |
| | GAGGCAGGGTGTTTCGCTCAGCACAACCCCAGTGT | |
| | AGATCAGGCTGATGAGTCACTGCAACCCCCATGGG | |
| | CGACCATGGCAGTGGCTGCGTTGGCGGCCTGCCCA | |
| | TGGAGAAATCCATGGGACGCTCTAATTCTGACATG | |
| | GTGTGAAGTGCCTATTGAGCTAACTGGTAGTCCTC | |
| | CGGCCCCTGATTGCGGCTAATCCTAACTGCGGAGC | |
| | ACATGCTCACAAACCAGTGGGTGGTGTGTCGTAAC | |
| | GGGCAACTCTGCAGCGGAACCGACTACTTTGGGTG | |
| | TCCGTGTTTCCTTTTATTCCTATATTGGCTGCTTAT | |
| | GGTGACAATCAAAAAGTTGTTACCATATAGCTATT | |
| | GGATTGGCCATCCGGTGTGCAACAGGGCAACTGTT | |
| | TACCTATTTATTGGTTTTGTACCATTATCACTGAAG | |
| | TCTGTGATCACTCTCAAATTCATTTTGACCCTCAAC | |
| | ACAATCAAACGCCACCATGGGATGGAGCTGTATCA | |
| | TCCTCTTCTTGGTAGCAACAGCTACCGGTGTGCAC | |
| | TCCCAGGTGCAATTGGTGCAGTCTGGCGCTGAAGT | |
| | TAAGAAGCCTGGGAGTTCAGTGAAGGTCTCCTGCA | |
| | AGGCTTCGGGATACGCCTTCAGCTATTCTTGGATC | |
| | AATTGGGTGCGGCAGGCGCCTGGACAAGGGCTCG | |
| | AGTGGATGGGACGGATCTTTCCCGGCGATGGGGAT | |
| | ACTGACTACAATGGGAAATTCAAGGGCAGAGTCA | |
| | CAATTACCGCCGACAAATCCACTAGCACAGCCTAT | |
| | ATGGAGCTGAGCAGCCTGAGATCTGAGGACACGG | |
| | CCGTGTATTACTGTGCAAGAAATGTCTTTGATGGT | |
| | TACTGGCTTGTTTACTGGGGCCAGGGAACCCTGGT | |
| | CACCGTCTCCAGCGCTAGCACCAAGGGCCCCTCCG | |
| | TGTTCCCCCTGGCCCCCAGCAGCAAGAGCACCAGC | |
| | GGCGGCACAGCCGCTCTGGGCTGCCTGGTCAAGGA | |
| | CTACTTCCCCGAGCCCGTGACCGTGTCCTGGAACA | |
| | GCGGAGCCCTGACCTCCGGCGTGCACACCTTCCCC | |
| | GCCGTGCTGCAGAGTTCTGGCCTGTATAGCCTGAG | |
| | CAGCGTGGTCACCGTGCCTTCTAGCAGCCTGGGCA | |
| | CCCAGACCTACATCTGCAACGTGAACCACAAGCCC | |
| | AGCAACACCAAGGTGGACAAGAAGGTGGAGCCCA | |
| | AGAGCTGCGGAGGGGGCGGATCCTTCTGGGTGCTG | |
| | GTGGTGGTGGGCGGCGTGCTGGCCTGCTACAGCCT | |
| | GCTGGTGACCGTGGCCTTCATCATCTTCTGGGTGA | |
| | GGAGCAAGAGGAGCAGGCTGCTGCACAGCGACTA | |
| | CATGAACATGACCCCCAGGAGGCCCGGCCCCACC | |
| | AGGAAGCACTACCAGCCCTACGCCCCCCCCAGGG | |
| | ACTTCGCCGCCTACAGGAGCAGGGTGAAGTTCAGC | |
| | AGGAGCGCCGACGCCCCCGCCTACCAGCAGGGCC | |
| | AGAACCAGCTGTATAACGAGCTGAACCTGGGCAG | |
| | GAGGGAGGAGTACGACGTGCTGGACAAGAGGAGG | |
| | GGCAGGGACCCCGAGATGGGCGGCAAGCCCAGGA | |
| | GGAAGAACCCCCAGGAGGGCCTGTATAACGAGCT | |
| | GCAGAAGGACAAGATGGCCGAGGCCTACAGCGAG | |
| | ATCGGCATGAAGGGCGAGAGGAGGAGGGGCAAGG | |
| | GCCACGACGGCCTGTACCAGGGCCTGAGCACCGCC | |
| | ACCAAGGACACCTACGACGCCCTGCACATGCAGG | |
| | CCCTGCCCCCCAGG | |
| Anti-CD20-Fab-VL | GATATCGTGATGACCCAGACTCCACTCTCCCTGCC | 23 |
| | CGTCACCCCTGGAGAGCCCGCCAGCATTAGCTGCA | |
| | GGTCTAGCAAGAGCCTCTTGCACAGCAATGGCATC | |
| | ACTTATTTGTATTGGTACCTGCAAAAGCCAGGGCA | |
| | GTCTCCACAGCTCCTGATTTATCAAATGTCCAACCT | |
| | TGTCTCTGGCGTCCCTGACCGGTTCTCCGGATCCG | |
| | GGTCAGGCACTGATTTCACACTGAAAATCAGCAGG | |
| | GTGGAGGCTGAGGATGTTGGAGTTTATTACTGCGC | |
| | TCAGAATCTAGAACTTCCTTACACCTTCGGCGGAG | |
| | GGACCAAGGTGGAGATCAAA | |
| Fab CL | CGTACGGTGGCTGCACCATCTGTCTTCATCTTCCCG | 24 |
| | CCATCTGATGAGCAGTTGAAATCTGGAACTGCCTC | |
| | TGTTGTGTGCCTGCTGAATAACTTCTATCCCAGAG | |
| | AGGCCAAAGTACAGTGGAAGGTGGATAACGCCCT | |
| | CCAATCGGGTAACTCCCAGGAGAGTGTCACAGAG | |
| | CAGGACAGCAAGGACAGCACCTACAGCCTCAGCA | |
| | GCACCCTGACGCTGAGCAAAGCAGACTACGAGAA | |
| | ACACAAAGTCTACGCCTGCGAAGTCACCCATCAGG | |
| | GCCTGAGCTCGCCCGTCACAAAGAGCTTCAACAGG | |
| | GGAGAGTGTTAG | |
| Anti-CD20-Fab-VH | CAGGTGCAATTGGTGCAGTCTGGCGCTGAAGTTAA | 25 |
| | GAAGCCTGGGAGTTCAGTGAAGGTCTCCTGCAAGG | |
| | CTTCGGGATACGCCTTCAGCTATTCTTGGATCAATT | |
| | GGGTGCGGCAGGCGCCTGGACAAGGGCTCGAGTG | |
| | GATGGGACGGATCTTTCCCGGCGATGGGGATACTG | |
| | ACTACAATGGGAAATTCAAGGGCAGAGTCACAAT | |
| | TACCGCCGACAAATCCACTAGCACAGCCTATATGG | |
| | AGCTGAGCAGCCTGAGATCTGAGGACACGGCCGT | |
| | GTATTACTGTGCAAGAAATGTCTTTGATGGTTACT | |
| | GGCTTGTTTACTGGGGCCAGGGAACCCTGGTCACC | |
| | GTCTCCAGC | |
| Fab CH1 | GCTAGCACCAAGGGCCCCTCCGTGTTCCCCCTGGC | 26 |
| | CCCCAGCAGCAAGAGCACCAGCGGCGGCACAGCC | |
| | GCTCTGGGCTGCCTGGTCAAGGACTACTTCCCCGA | |
| | GCCCGTGACCGTGTCCTGGAACAGCGGAGCCCTGA | |
| | CCTCCGGCGTGCACACCTTCCCCGCCGTGCTGCAG | |
| | AGTTCTGGCCTGTATAGCCTGAGCAGCGTGGTCAC | |
| | CGTGCCTTCTAGCAGCCTGGGCACCCAGACCTACA | |
| | TCTGCAACGTGAACCACAAGCCCAGCAACACCAA | |
| | GGTGGACAAGAAGGTGGAGCCCAAGAGCTGC | |
| IRES EV71 | CCCGAAGTAACTTAGAAGCTGTAAATCAACGATCA | 27 |
| internal | ATAGCAGGTGTGGCACACCAGTCATACCTTGATCA | |
| ribosomal | AGCACTTCTGTTTCCCCGGACTGAGTATCAATAGG | |
| entry side | CTGCTCGCGCGGCTGAAGGAGAAAACGTTCGTTAC | |
| | CCGACCAACTACTTCGAGAAGCTTAGTACCACCAT | |
| | GAACGAGGCAGGGTGTTTCGCTCAGCACAACCCCA | |
| | GTGTAGATCAGGCTGATGAGTCACTGCAACCCCCA | |
| | TGGGCGACCATGGCAGTGGCTGCGTTGGCGGCCTG | |
| | CCCATGGAGAAATCCATGGGACGCTCTAATTCTGA | |
| | CATGGTGTGAAGTGCCTATTGAGCTAACTGGTAGT | |
| | CCTCCGGCCCCTGATTGCGGCTAATCCTAACTGCG | |
| | GAGCACATGCTCACAAACCAGTGGGTGGTGTGTCG | |
| | TAACGGGCAACTCTGCAGCGGAACCGACTACTTTG | |
| | GGTGTCCGTGTTTCCTTTTATTCCTATATTGGCTGC | |
| | TTATGGTGACAATCAAAAAGTTGTTACCATATAGC | |
| | TATTGGATTGGCCATCCGGTGTGCAACAGGGCAAC | |
| | TGTTTACCTATTTATTGGTTTTGTACCATTATCACT | |
| | GAAGTCTGTGATCACTCTCAAATTCATTTTGACCCT | |
| | CAACACAATCAAAC | |
| G4S linker | GGAGGGGGCGGATCC | 28 |
| CD28ATD | TTTTGGGTGCTGGTGGTGGTTGGTGGAGTCCTGGC | 29 |
| | TTGCTATAGCTTGCTAGTAACAGTGGCCTTTATTAT | |
| | TTTCTGGGTG | |
| CD28CSD | AGGAGTAAGAGGAGCAGGCTCCTGCACAGTGACT | 30 |
| | ACATGAACATGACTCCCCGCCGCCCCGGGCCCACC | |
| | CGCAAGCATTACCAGCCCTATGCCCCACCACGCGA | |
| | CTTCGCAGCCTATCGCTCC | |
| CD3zSSD | AGAGTGAAGTTCAGCAGGAGCGCAGACGCCCCCG | 31 |
| | CGTACCAGCAGGGCCAGAACCAGCTCTATAACGA | |
| | GCTCAATCTAGGACGAAGAGAGGAGTACGATGTTT | |
| | TGGACAAGAGACGTGGCCGGGACCCTGAGATGGG | |
| | GGGAAAGCCGAGAAGGAAGAACCCTCAGGAAGGC | |
| | CTGTACAATGAACTGCAGAAAGATAAGATGGCGG | |
| | AGGCCTACAGTGAGATTGGGATGAAAGGCGAGCG | |
| | CCGGAGGGGCAAGGGGCACGATGGCCTTTACCAG | |
| | GGTCTCAGTACAGCCACCAAGGACACCTACGACGC | |
| | CCTTCACATGCAGGCCCTGCCCCCTCGC | |
| CD28ATD-CD28CSD-CD3zSSD | TTCTGGGTGCTGGTGGTGGTGGGCGGCGTGCTGGC | 32 |
| | CTGCTACAGCCTGCTGGTGACCGTGGCCTTCATCA | |
| | TCTTCTGGGTGAGGAGCAAGAGGAGCAGGCTGCT | |
| | GCACAGCGACTACATGAACATGACCCCCAGGAGG | |
| | CCCGGCCCCACCAGGAAGCACTACCAGCCCTACGC | |
| | CCCCCCCAGGGACTTCGCCGCCTACAGGAGCAGGG | |
| | TGAAGTTCAGCAGGAGCGCCGACGCCCCCGCCTAC | |
| | CAGCAGGGCCAGAACCAGCTGTATAACGAGCTGA | |
| | ACCTGGGCAGGAGGGAGGAGTACGACGTGCTGGA | |
| | CAAGAGGAGGGGCAGGGACCCCGAGATGGGCGGC | |
| | AAGCCCAGGAGGAAGAACCCCCAGGAGGGCCTGT | |
| | ATAACGAGCTGCAGAAGGACAAGATGGCCGAGGC | |
| | CTACAGCGAGATCGGCATGAAGGGCGAGAGGAGG | |
| | AGGGGCAAGGGCCACGACGGCCTGTACCAGGGCC | |
| | TGAGCACCGCCACCAAGGACACCTACGACGCCCTG | |
| | CACATGCAGGCCCTGCCCCCCAGG | |
| T2A linker | TCCGGAGAGGGCAGAGGAAGTCTTCTAACATGCG | 33 |
| | GTGACGTGGAGGAGAATCCCGGCCCTAGG | |
| eGFP | GTGAGCAAGGGCGAGGAGCTGTTCACCGGGGTGG | 34 |
| | TGCCCATCCTGGTCGAGCTGGACGGCGACGTAAAC | |
| | GGCCACAAGTTCAGCGTGTCCGGCGAGGGCGAGG | |
| | GCGATGCCACCTACGGCAAGCTGACCCTGAAGTTC | |
| | ATCTGCACCACCGGCAAGCTGCCCGTGCCCTGGCC | |
| | CACCCTCGTGACCACCCTGACCTACGGCGTGCAGT | |
| | GCTTCAGCCGCTACCCCGACCACATGAAGCAGCAC | |
| | GACTTCTTCAAGTCCGCCATGCCCGAAGGCTACGT | |
| | CCAGGAGCGCACCATCTTCTTCAAGGACGACGGCA | |
| | ACTACAAGACCCGCGCCGAGGTGAAGTTCGAGGG | |
| | CGACACCCTGGTGAACCGCATCGAGCTGAAGGGC | |
| | ATCGACTTCAAGGAGGACGGCAACATCCTGGGGC | |
| | ACAAGCTGGAGTACAACTACAACAGCCACAACGT | |
| | CTATATCATGGCCGACAAGCAGAAGAACGGCATC | |
| | AAGGTGAACTTCAAGATCCGCCACAACATCGAGG | |
| | ACGGCAGCGTGCAGCTCGCCGACCACTACCAGCA | |
| | GAACACCCCCATCGGCGACGGCCCCGTGCTGCTGC | |
| | CCGACAACCACTACCTGAGCACCCAGTCCGCCCTG | |
| | AGCAAAGACCCCAACGAGAAGCGCGATCACATGG | |
| | TCCTGCTGGAGTTCGTGACCGCCGCCGGGATCACT | |
| | CTCGGCATGGACGAGCTGTACAAGTGA | |
| Anti-CD2O-Fab-CD28ATD-CD28CSD- | ATGGGATGGAGCTGTATCATCCTCTTCTTGGTAGC | 35 |
| CD3zSSD-eGFP | AACAGCTACCGGTGTGCATTCCGATATCGTGATGA | |
| | CCCAGACTCCACTCTCCCTGCCCGTCACCCCTGGA | |
| | GAGCCCGCCAGCATTAGCTGCAGGTCTAGCAAGA | |
| | GCCTCTTGCACAGCAATGGCATCACTTATTTGTATT | |
| | GGTACCTGCAAAAGCCAGGGCAGTCTCCACAGCTC | |
| | CTGATTTATCAAATGTCCAACCTTGTCTCTGGCGTC | |
| | CCTGACCGGTTCTCCGGATCCGGGTCAGGCACTGA | |
| | TTTCACACTGAAAATCAGCAGGGTGGAGGCTGAG | |
| | GATGTTGGAGTTTATTACTGCGCTCAGAATCTAGA | |
| | ACTTCCTTACACCTTCGGCGGAGGGACCAAGGTGG | |
| | AGATCAAACGTACGGTGGCTGCACCATCTGTCTTC | |
| | ATCTTCCCGCCATCTGATGAGCAGTTGAAATCTGG | |
| | AACTGCCTCTGTTGTGTGCCTGCTGAATAACTTCTA | |
| | TCCCAGAGAGGCCAAAGTACAGTGGAAGGTGGAT | |
| | AACGCCCTCCAATCGGGTAACTCCCAGGAGAGTGT | |
| | CACAGAGCAGGACAGCAAGGACAGCACCTACAGC | |
| | CTCAGCAGCACCCTGACGCTGAGCAAAGCAGACT | |
| | ACGAGAAACACAAAGTCTACGCCTGCGAAGTCAC | |
| | CCATCAGGGCCTGAGCTCGCCCGTCACAAAGAGCT | |
| | TCAACAGGGGAGAGTGTTAGAATAGAATTCCCCG | |
| | AAGTAACTTAGAAGCTGTAAATCAACGATCAATAG | |
| | CAGGTGTGGCACACCAGTCATACCTTGATCAAGCA | |
| | CTTCTGTTTCCCCGGACTGAGTATCAATAGGCTGCT | |
| | CGCGCGGCTGAAGGAGAAAACGTTCGTTACCCGA | |
| | CCAACTACTTCGAGAAGCTTAGTACCACCATGAAC | |
| | GAGGCAGGGTGTTTCGCTCAGCACAACCCCAGTGT | |
| | AGATCAGGCTGATGAGTCACTGCAACCCCCATGGG | |
| | CGACCATGGCAGTGGCTGCGTTGGCGGCCTGCCCA | |
| | TGGAGAAATCCATGGGACGCTCTAATTCTGACATG | |
| | GTGTGAAGTGCCTATTGAGCTAACTGGTAGTCCTC | |
| | CGGCCCCTGATTGCGGCTAATCCTAACTGCGGAGC | |
| | ACATGCTCACAAACCAGTGGGTGGTGTGTCGTAAC | |
| | GGGCAACTCTGCAGCGGAACCGACTACTTTGGGTG | |
| | TCCGTGTTTCCTTTTATTCCTATATTGGCTGCTTAT | |
| | GGTGACAATCAAAAAGTTGTTACCATATAGCTATT | |
| | GGATTGGCCATCCGGTGTGCAACAGGGCAACTGTT | |
| | TACCTATTTATTGGTTTTGTACCATTATCACTGAAG | |
| | TCTGTGATCACTCTCAAATTCATTTTGACCCTCAAC | |
| | ACAATCAAACGCCACCATGGGATGGAGCTGTATCA | |
| | TCCTCTTCTTGGTAGCAACAGCTACCGGTGTGCAC | |
| | TCCCAGGTGCAATTGGTGCAGTCTGGCGCTGAAGT | |
| | TAAGAAGCCTGGGAGTTCAGTGAAGGTCTCCTGCA | |
| | AGGCTTCGGGATACGCCTTCAGCTATTCTTGGATC | |
| | AATTGGGTGCGGCAGGCGCCTGGACAAGGGCTCG | |
| | AGTGGATGGGACGGATCTTTCCCGGCGATGGGGAT | |
| | ACTGACTACAATGGGAAATTCAAGGGCAGAGTCA | |
| | CAATTACCGCCGACAAATCCACTAGCACAGCCTAT | |
| | ATGGAGCTGAGCAGCCTGAGATCTGAGGACACGG | |
| | CCGTGTATTACTGTGCAAGAAATGTCTTTGATGGT | |
| | TACTGGCTTGTTTACTGGGGCCAGGGAACCCTGGT | |
| | CACCGTCTCCAGCGCTAGCACCAAGGGCCCCTCCG | |
| | TGTTCCCCCTGGCCCCCAGCAGCAAGAGCACCAGC | |
| | GGCGGCACAGCCGCTCTGGGCTGCCTGGTCAAGGA | |
| | CTACTTCCCCGAGCCCGTGACCGTGTCCTGGAACA | |
| | GCGGAGCCCTGACCTCCGGCGTGCACACCTTCCCC | |
| | GCCGTGCTGCAGAGTTCTGGCCTGTATAGCCTGAG | |
| | CAGCGTGGTCACCGTGCCTTCTAGCAGCCTGGGCA | |
| | CCCAGACCTACATCTGCAACGTGAACCACAAGCCC | |
| | AGCAACACCAAGGTGGACAAGAAGGTGGAGCCCA | |
| | AGAGCTGCGGAGGGGGCGGATCCTTCTGGGTGCTG | |
| | GTGGTGGTGGGCGGCGTGCTGGCCTGCTACAGCCT | |
| | GCTGGTGACCGTGGCCTTCATCATCTTCTGGGTGA | |
| | GGAGCAAGAGGAGCAGGCTGCTGCACAGCGACTA | |
| | CATGAACATGACCCCCAGGAGGCCCGGCCCCACC | |
| | AGGAAGCACTACCAGCCCTACGCCCCCCCCAGGG | |
| | ACTTCGCCGCCTACAGGAGCAGGGTGAAGTTCAGC | |
| | AGGAGCGCCGACGCCCCCGCCTACCAGCAGGGCC | |
| | AGAACCAGCTGTATAACGAGCTGAACCTGGGCAG | |
| | GAGGGAGGAGTACGACGTGCTGGACAAGAGGAGG | |
| | GGCAGGGACCCCGAGATGGGCGGCAAGCCCAGGA | |
| | GGAAGAACCCCCAGGAGGGCCTGTATAACGAGCT | |
| | GCAGAAGGACAAGATGGCCGAGGCCTACAGCGAG | |
| | ATCGGCATGAAGGGCGAGAGGAGGAGGGGCAAGG | |
| | GCCACGACGGCCTGTACCAGGGCCTGAGCACCGCC | |
| | ACCAAGGACACCTACGACGCCCTGCACATGCAGG | |
| | CCCTGCCCCCCAGGTCCGGAGAGGGCAGAGGAAG | |
| | TCTTCTAACATGCGGTGACGTGGAGGAGAATCCCG | |
| | GCCCTAGGGTGAGCAAGGGCGAGGAGCTGTTCAC | |
| | CGGGGTGGTGCCCATCCTGGTCGAGCTGGACGGCG | |
| | ACGTAAACGGCCACAAGTTCAGCGTGTCCGGCGA | |
| | GGGCGAGGGCGATGCCACCTACGGCAAGCTGACC | |
| | CTGAAGTTCATCTGCACCACCGGCAAGCTGCCCGT | |
| | GCCCTGGCCCACCCTCGTGACCACCCTGACCTACG | |
| | GCGTGCAGTGCTTCAGCCGCTACCCCGACCACATG | |
| | AAGCAGCACGACTTCTTCAAGTCCGCCATGCCCGA | |
| | AGGCTACGTCCAGGAGCGCACCATCTTCTTCAAGG | |
| | ACGACGGCAACTACAAGACCCGCGCCGAGGTGAA | |
| | GTTCGAGGGCGACACCCTGGTGAACCGCATCGAGC | |
| | TGAAGGGCATCGACTTCAAGGAGGACGGCAACAT | |
| | CCTGGGGCACAAGCTGGAGTACAACTACAACAGC | |
| | CACAACGTCTATATCATGGCCGACAAGCAGAAGA | |
| | ACGGCATCAAGGTGAACTTCAAGATCCGCCACAAC | |
| | ATCGAGGACGGCAGCGTGCAGCTCGCCGACCACT | |
| | ACCAGCAGAACACCCCCATCGGCGACGGCCCCGT | |
| | GCTGCTGCCCGACAACCACTACCTGAGCACCCAGT | |
| | CCGCCCTGAGCAAAGACCCCAACGAGAAGCGCGA | |
| | TCACATGGTCCTGCTGGAGTTCGTGACCGCCGCCG | |
| | GGATCACTCTCGGCATGGACGAGCTGTACAAGTGA | |

TABLE 4
Anti-CEA (98/99) scFv sequences

| | | SEQ |
|---|---|---|
| | | ID |
| Construct | Protein Sequence | NO |
| Anti-CEA CDR H1 | EFGMN | 36 |
| Kabat | | |
| Anti-CEA CDR H2 | WINTKTGEATYVEEFKG | 37 |
| Kabat | | |
| Anti-CEA CDR H3 | WDFAYYVEAMDY | 38 |
| Kabat | | |
| Anti-CEA CDR Ll | KASAAVGTYVA | 39 |
| Kabat | | |
| Anti-CEA CDR L2 | SASYRKR | 40 |
| Kabat | | |
| Anti-CEA CDR L3 | HQYYTYPLFT | 41 |
| Kabat | | |
| Anti-CEA (98/99)-scFv-CD28ATD- | QVQLVQSGAEVKKPGASVKVSCKASGYTFTEFGMN | 42 |
| CD28CSD-CD3zSSD fusion | WVRQAPGQGLEWMGWINTKTGEATYVEEFKGRVTF | |
| | TTDTSTSTAYMELRSLRSDDTAVYYCARWDFAYYV | |
| | EAMDYWGQGTTVTVSSGGGGSGGGGSGGGGSGGG | |
| | GSDIQMTQSPSSLSASVGDRVTITCKASAAVGTYVA | |
| | WYQQKPGKAPKLLIYSASYRKRGVPSRFSGSGSGTD | |
| | FTLTISSLQPEDFATYYCHQYYTYPLFTFGQGTKLEIK | |
| | RTGGGGSFWVLVVVGGVLACYSLLVTVAFIIFWVRS | |
| | KRSRLLHSDYMNMTPRRPGPTRKHYQPYAPPRDFAA | |
| | YRSRVKFSRSADAPAYQQGQNQLYNELNLGRREEY | |
| | DVLDKRRGRDPEMGGKPRRKNPQEGLYNELQKDK | |
| | MAEAYSEIGMKGERRRGKGHDGLYQGLSTATKDTY | |
| | DALHMQALPPR | |
| Anti-CEA (98/99)-scFv | QVQLVQSGAEVKKPGASVKVSCKASGYTFTEFGMN | 43 |
| | WVRQAPGQGLEWMGWINTKTGEATYVEEFKGRVTF | |
| | TTDTSTSTAYMELRSLRSDDTAVYYCARWDFAYYV | |
| | EAMDYWGQGTTVTVSSGGGGSGGGGSGGGGSGGG | |
| | GSDIQMTQSPSSLSASVGDRVTITCKASAAVGTYVA | |
| | WYQQKPGKAPKLLIYSASYRKRGVPSRFSGSGSGTD | |
| | FTLTISSLQPEDFATYYCHQYYTYPLFTFGQGTKLEIK | |
| | RT | |
| Anti-CEA (98/99)-VH | QVQLVQSGAEVKKPGASVKVSCKASGYTFTEFGMN | 44 |
| | WVRQAPGQGLEWMGWINTKTGEATYVEEFKGRVTF | |
| | TTDTSTSTAYMELRSLRSDDTAVYYCARWDFAYYV | |
| | EAMDYWGQGTTVTVSS | |
| Anti-CEA (98/99)-VL | DIQMTQSPSSLSASVGDRVTITCKASAAVGTYVAWY | 45 |
| | QQKPGKAPKLLIYSASYRKRGVPSRFSGSGSGTDFTL | |
| | TISSLQPEDFATYYCHQYYTYPLFTFGQGTKLEIKRT | |

TABLE 5
Anti-WT1 (11D06) exemplary Fab sequences

| | | SEQ |
|---|---|---|
| | | ID |
| Construct | Protein Sequence | NO |
| Anti-WT1 (11D06) | GGTFSSYAIS | 46 |
| CDR H1 Kabat | | |
| Anti-WT1 (11D06) | GIIPIFGTANYAQKFQG | 47 |
| CDR H2 Kabat | | |
| Anti-WT1 (11D06) | SIELWWGGFDY | 48 |
| CDR H3 Kabat | | |
| Anti-WT1 (11D06) | RASQSISSWLA | 49 |
| CDR Li Kabat | | |
| Anti-WT1 (11D06) | DASSLES | 50 |
| CDR L2 Kabat | | |
| Anti-WT1 (11D06) | QQYEDYTT | 51 |
| CDR L3 Kabat | | |
| Anti-WT1 (11D06)-Fab-heavy chain-CD28ATD- | QVQLVQSGAEVKKPGSSVKVSCKASGGTFSSYAISW | 52 |
| CD28CSD-CD3zSSD fusion | VRQAPGQGLEWMGGIIPIFGTANYAQKFQGRVTITA | |
| | DKSTSTAYMELSSLRSEDTAVYYCARSIELWWGGFD | |
| | YWGQGTTVTVSSASTKGPSVFPLAPSSKSTSGGTAAL | |
| | GCLVKDYFPEPVTVSWNSGALTSGVHTFPAVLQSSG | |
| | LYSLSSVVTVPSSSLGTQTYICNVNHKPSNTKVDKKV | |
| | EPKSCGGGGSFWVLVVVGGVLACYSLLVTVAFIIFW | |
| | VRSKRSRLLHSDYMNMTPRRPGPTRKHYQPYAPPRD | |
| | FAAYRSRVKFSRSADAPAYQQGQNQLYNELNLGRR | |
| | EEYDVLDKRRGRDPEMGGKPRRKNPQEGLYNELQK | |
| | DKMAEAYSEIGMKGERRRGKGHDGLYQGLSTATKD | |
| | TYDALHMQALPPR | |
| Anti-WT1 (11D06)-Fab-heavy chain | QVQLVQSGAEVKKPGSSVKVSCKASGGTFSSYAISW | 53 |
| | VRQAPGQGLEWMGGIIPIFGTANYAQKFQGRVTITA | |
| | DKSTSTAYMELSSLRSEDTAVYYCARSIELWWGGFD | |
| | YWGQGTTVTVSSLRSEDTAVYYCARNVFDGYWLVY | |
| | WGQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKVE | |
| | PKSC | |
| Anti-WT1 (11D06)-light chain | DIQMTQSPSTLSASVGDRVTITCRASQSISSWLAWYQ | 54 |
| | QKPGKAPKLLIYDASSLESGVPSRFSGSGSGTEFTLTI | |
| | GSLQPDDFATYYCQQYEDYTTFGQGTKVEIKTVAAP | |
| | SVFIFPPSDEQLKSGTASVVCLLNNFYPREAKVQWKV | |
| | DNALQSGNSQESVTEQDSKDSTYSLSSTLTLSKADYE | |
| | KHKVYACEVTHQGLSSPVTKSFNRGEC | |
| Anti-WT1 (11D06)-VL | DIQMTQSPSTLSASVGDRVTITCRASQSISSWLAWYQ | 55 |
| | QKPGKAPKLLIYDASSLESGVPSRFSGSGSGTEFTLTI | |
| | GSLQPDDFATYYCQQYEDYTTFGQGTKVEIK | |
| Anti-WT1 (11D06)-VH | QVQLVQSGAEVKKPGSSVKVSCKASGGTFSSYAISW | 56 |
| | VRQAPGQGLEWMGGIIPIFGTANYAQKFQGRVTITA | |
| | DKSTSTAYMELSSLRSEDTAVYYCARSIELWWGGFD | |
| | YWGQGTTVTVSS | |

TABLE 4
Anti-WT1 (33H09) exemplary scFv sequences

| | | SEQ |
|---|---|---|
| | | ID |
| Construct | Protein Sequence | NO |
| Anti-WT1 (33H09) | see Table 5 | 46 |
| CDR H1 Kabat | | |
| Anti-WT1 (33H09) | see Table 5 | 47 |
| CDR H2 Kabat | | |
| Anti-WT1 (33H09) | GSYDLFSLDY | 57 |
| CDR H3 Kabat | | |
| Anti-WT1 (33H09) | see Table 5 | 49 |
| CDR Ll Kabat | | |
| Anti-WT1 (33H09) | see Table 5 | 50 |
| CDR L2 Kabat | | |
| Anti-WT1 (33H09) | QQYYDGIT | 58 |
| CDR L3 Kabat | | |
| Anti-WT1 (33H09)-scFv-CD28ATD- | QVQLVQSGAEVKKPGSSVKVSCKASGGTFSSYAISW | 59 |
| CD28CSD-CD3zSSD fusion | VRQAPGQGLEWMGGIIPIFGTANYAQKFQGRVTITA | |
| | DKSTSTAYMELSSLRSEDTAVYYCARGSYDLFSLDY | |
| | WGQGTTVTVSSGGGGSGGGGSGGGGSGGGGSDIQM | |
| | TQSPSSLSASVGDRVTITCKASAAVGTYVAWYQQKP | |
| | GKAPKLLIYSASYRKRGVPSRFSGSGSGTDFTLTISSL | |
| | QPEDFATYYCHQYYTYPLFTFGQGTKLEIKRTGGGG | |
| | SFWVLVVVGGVLACYSLLVTVAFIIFWVRSKRSRLL | |
| | HSDYMNMTPRRPGPTRKHYQPYAPPRDFAAYRSRV | |
| | KFSRSADAPAYQQGQNQLYNELNLGRREEYDVLDK | |
| | RRGRDPEMGGKPRRKNPQEGLYNELQKDKMAEAYS | |
| | EIGMKGERRRGKGHDGLYQGLSTATKDTYDALHMQ | |
| | ALPPR | |
| Anti-WT1 (33H09)-scFv | QVQLVQSGAEVKKPGSSVKVSCKASGGTFSSYAISW | 60 |
| | VRQAPGQGLEWMGGIIPIFGTANYAQKFQGRVTITA | |
| | DKSTSTAYMELSSLRSEDTAVYYCARGSYDLFSLDY | |
| | WGQGTTVTVSSGGGGSGGGGSGGGGSGGGGSDIQM | |
| | TQSPSTLSASVGDRVTITCRASQSISSWLAWYQQKPG | |
| | KAPKLLIYDASSLESGVPSRFSGSGSGTEFTLTISS | |
| | LQPDDFATYYCQQYYDGITFGQGTKVEIK | |
| Anti-WT1 (33H09)-VH | QVQLVQSGAEVKKPGSSVKVSCKASGGTFSSYAISW | 61 |
| | VRQAPGQGLEWMGGIIPIFGTANYAQKFQGRVTITA | |
| | DKSTSTAYMELSSLRSEDTAVYYCARGSYDLFSLDY | |
| | WGQGTTVTVSS | |
| Anti-WT1 (33H09)-VL | DIQMTQSPSTLSASVGDRVTITCRASQSISSWLAWYQ | 62 |
| | QKPGKAPKLLIYDASSLESGVPSRFSGSGSGTEFTLTI | |
| | SSLQPDDFATYYCQQYYDGITFGQGTKVEIK | |

TABLE 14

| | | SEQ |
|---|---|---|
| | | ID |
| Construct | Amino acid sequence | NO |
| Human CD3z | MKWKALFTAAILQAQLPITEAQSFGLLDPKLCYLLD | 63 |
| | GILFIYGVILTALFLRVKFSRSADAPAYQQGQNQLYN | |
| | ELNLGRREEYDVLDKRRGRDPEMGGKPQRRKNPQE | |
| | GLYNELQKDKMAEAYSEIGMKGERRRGKGHDGLYQ | |
| | GLSTATKDTYDALHMQALPPR | |
| Human CD3z | ATGAAGTGGAAGGCGCTTTTCACCGCGGCCATCCT | 64 |
| | GCAGGCACAGTTGCCGATTACAGAGGCACAGAGC | |
| | TTTGGCCTGCTGGATCCCAAACTCTGCTACCTGCTG | |
| | GATGGAATCCTCTTCATCTATGGTGTCATTCTCACT | |
| | GCCTTGTTCCTGAGAGTGAAGTTCAGCAGGAGCGC | |
| | AGAGCCCCCCGCGTACCAGCAGGGCCAGAACCAG | |
| | CTCTATAACGAGCTCAATCTAGGACGAAGAGAGG | |
| | AGTACGATGTTTTGGACAAGAGACGTGGCCGGGA | |
| | CCCTGAGATGGGGGGAAAGCCGAGAAGGAAGAAC | |
| | CCTCAGGAAGGCCTGTACAATGAACTGCAGAAAG | |
| | ATAAGATGGCGGAGGCCTACAGTGAGATTGGGAT | |
| | GAAAGGCGAGCGCCGGAGGGGCAAGGGGCACGAT | |
| | GGCCTTTACCAGGGTCTCAGTACAGCCACCAAGGA | |
| | CACCTACGACGCCCTTCACATGCAGGCCCTGCCCC | |
| | CTCGCTAA | |
| Murine CD3z | MKWKVSVLACILHVRFPGAEAQSFGLLDPKLCYLLD | 65 |
| | GILFIYGVIITALYLRAKFSRSAETAANLQDPNQLYNE | |
| | LNLGRREEYDVLEKKRARDPEMGGKQQRRRNPQEG | |
| | VYNALQKDKMAEAYSEIGTKGERRRGKGHDGLYQG | |
| | LSTATKDTYDALHMQTLAPR | |
| Murine CD3z | ATGAAGTGGAAAGTGTCTGTTCTCGCCTGCATCCT | 66 |
| | CCACGTGCGGTTCCCAGGAGCAGAGGCACAGAGC | |
| | TTTGGTCTGCTGGATCCCAAACTCTGCTACTTGCTA | |
| | GATGGAATCCTCTTCATCTACGGAGTCATCATCAC | |
| | AGCCCTGTACCTGAGAGCAAAATTCAGCAGGAGT | |
| | GCAGAGACTGCTGCCAACCTGCAGGACCCCAACC | |
| | AGCTCTACAATGAGCTCAATCTAGGGCGAAGAGA | |
| | GGAATATGACGTCTTGGAGAAGAAGCGGGCTCGG | |
| | GATCCAGAGATGGGAGGCAAACAGCAGAGGAGGA | |
| | GGAACCCCCAGGAAGGCGTATACAATGCACTGCA | |
| | GAAAGACAAGATGGCAGAAGCCTACAGTGAGATC | |
| | GGCACAAAAGGCGAGAGGCGGAGAGGCAAGGGG | |
| | CACGATGGCCTTTACCAGGGTCTCAGCACTGCCAC | |
| | CAAGGACACCTATGATGCCCTGCATATGCAGACCC | |
| | TGGCCCCTCGCTAA | |
| Human CD28 | ATGCTGCGCCTGCTGCTGGCGCTGAACCTGTTTCC | 67 |
| | GAGCATTCAGGTGACCGGCAACAAAATTCTGGTGA | |
| | AACAGAGCCCGATGCTGGTGGCGTATGATAACGC | |
| | GGTGAACCTGAGCTGCAAATATAGCTATAACCTGT | |
| | TTAGCCGCGAATTTCGCGCGAGCCTGCATAAAGGC | |
| | CTGGATAGCGCGGTGGAAGTGTGCGTGGTGTATGG | |
| | CAACTATAGCCAGCAGCTGCAGGTGTATAGCAAA | |
| | ACCGGCTTTAACTGCGATGGCAAACTGGGCAACGA | |
| | AAGCGTGACCTTTTATCTGCAGAACCTGTATGTGA | |
| | ACCAGACCGATATTTATTTTTGCAAAATTGAAGTG | |
| | ATGTATCCGCCGCCGTATCTGGATAACGAAAAAAG | |
| | CAACGGCACCATTATTCATGTGAAAGGCAAACATC | |
| | TGTGCCCGAGCCCGCTGTTTCCGGGCCCGAGCAAA | |
| | CCGTTTTGGGTGCTGGTGGTGGTGGGCGGCGTGCT | |
| | GGCGTGCTATAGCCTGCTGGTGACCGTGGCGTTTA | |
| | TTATTTTTTGGGTGCGCAGCAAACGCAGCCGCCTG | |
| | CTGCATAGCGATTATATGAACATGACCCCGCGCCG | |
| | CCCGGGCCCGACCCGCAAACATTATCAGCCGTATG | |
| | CGCCGCCGCGCGATTTTGCGGCGTATCGCAGC | |
| Human CD28 | MLRLLLALNLFPSIQVTGNKILVKQSPMLVAYDNAV | 68 |
| | NLSCKYSYNLFSREFRASLHKGLDSAVEVCVVYGNY | |
| | SQQLQVYSKTGFNCDGKLGNESVTFYLQNLYVNQT | |
| | DIYFCKIEVMYPPPYLDNEKSNGTIIHVKGKHLCPSPL | |
| | FPGPSKPFWVLVVVGGVLACYSLLVTVAFIIFWVRSK | |
| | RSRLLHSDYMNMTPRRPGPTRKHYQPYAPPRDFAAY | |
| | RS | |
| Murine CD28 | ATGACCCTGCGCCTGCTGTTTCTGGCGCTGAACTTT | 69 |
| | TTTAGCGTGCAGGTGACCGAAAACAAAATTCTGGT | |
| | GAAACAGAGCCCGCTGCTGGTGGTGGATAGCAAC | |
| | GAAGTGAGCCTGAGCTGCCGCTATAGCTATAACCT | |
| | GCTGGCGAAAGAATTTCGCGCGAGCCTGTATAAAG | |
| | GCGTGAACAGCGATGTGGAAGTGTGCGTGGGCAA | |
| | CGGCAACTTTACCTATCAGCCGCAGTTTCGCAGCA | |
| | ACGCGGAATTTAACTGCGATGGCGATTTTGATAAC | |
| | GAAACCGTGACCTTTCGCCTGTGGAACCTGCATGT | |
| | GAACCATACCGATATTTATTTTTGCAAAATTGAAT | |
| | TTATGTATCCGCCGCCGTATCTGGATAACGAACGC | |
| | AGCAACGGCACCATTATTCATATTAAAGAAAAACA | |
| | TCTGTGCCATACCCAGAGCAGCCCGAAACTGTTTT | |
| | GGGCGCTGGTGGTGGTGGCGGGCGTGCTGTTTTGC | |
| | TATGGCCTGCTGGTGACCGTGGCGCTGTGCGTGAT | |
| | TTGGACCAACAGCCGCCGCAACCGCCTGCTGCAGA | |
| | GCGATTATATGAACATGACCCCGCGCCGCCCGGGC | |
| | CTGACCCGCAAACCGTATCAGCCGTATGCGCCGGC | |
| | GCGCGATTTTGCGGCGTATCGCCCG | |
| Murine CD28 | MTLRLLFLALNFFSVQVTENKILVKQSPLLVVDSNEV | 70 |
| | SLSCRYSYNLLAKEFRASLYKGVNSDVEVCVGNGNF | |
| | TYQPQFRSNAEFNCDGDFDNETVTFRLWNLHVNHT | |
| | DIYFCKIEFMYPPPYLDNERSNGTIIHIKEKHLCHTQSS | |
| | PKLFWALVVVAGVLFCYGLLVTVALCVIWTNSRRN | |
| | RLLQSDYMNMTPRRPGLTRKPYQPYAPARDFAAYRP | |
| CD28 YMNM | YMNM | 71 |
| CD28 PYAP | PYAP | 72 |
| Signal peptide | ATMGWSCIILFLVATATGVHS | 73 |
| Signal peptide DNA sequence | ATGGGATGGAGCTGTATCATCCTCTTCTTGGTAGC | 74 |
| | AACAGCTACCGGTGTGCACTCC | |

1. A diagnostic assay for determining the presence of a tumor cell in a sample, the diagnostic assay comprising the steps of:
a) contacting the sample with a chimeric antigen receptor (CAR) expressing reporter T (CAR-T) cell wherein the reporter CAR-T cell comprises:
i. a CAR capable of specific binding to the tumor cell, wherein the CAR is operationally coupled to a response element;
ii. a reporter gene under the control of the response element; and
b) determining T cell activation by measuring the expression of the reporter gene to establish the presence of the tumor cell.

2. The diagnostic assay of claim 1, wherein the CAR comprises a target antigen binding moiety capable of specific binding to a tumor target antigen.

3. The diagnostic assay of claim 2, wherein the target antigen binding moiety is a Fab fragment.

4. The diagnostic assay of any one of claims 1 to 3, wherein the reporter CAR-T cell is a Jurkat cell.

5. The diagnostic assay of any one of claims 2 to 4, wherein the tumor target antigen is a cell surface antigen and/or receptor.

6. The diagnostic assay of any one of claims 2 or 5, wherein the tumor target antigen is selected from the group consisting of CD20, CD38, CD138, CEA, EGFR, Fo1R1, HER2, LeY, MCSP, STEAP1, TYRP1, and WT1, or a fragment thereof.

7. The diagnostic assay of any one of claims 2 to 6, wherein the tumor target antigen is a peptide bound to a molecule of the human major histocompatibility complex (MHC).

8. The diagnostic assay of any one of claims 1 to 7, wherein the CAR comprises at least one intracellular stimulatory signaling and/or co-stimulatory signaling domain.

9. The diagnostic assay of claim 8, wherein activation of the intracellular signaling and/or co-signaling domain leads to activation of the response element.

10. The diagnostic assay of any one of claims 1 to 9, wherein activation of the response element leads to expression of the reporter gene.

11. The diagnostic assay of any one of embodiments 1 to 10, wherein the response element is part of the NFAT pathway, the NF-κB pathway or the AP-1 pathway.

12. The diagnostic assay of any one of embodiments 1 to 11, wherein the reporter gene is coding for a luminescent protein.

13. The diagnostic assay of any one of embodiments 1 to 12, wherein the reporter gene is coding for green fluorescent protein (GFP) or luciferase.

14. The diagnostic assay of any one of claims 1 to 13, wherein the sample is a patient sample derived from an individual suffering from a disease, in particular wherein the disease is cancer.

15. A method for predicting the efficacy of an antitumor CAR-T cell treatment, comprising providing a sample from a subject having a tumor, and determining T cell activation by measuring the expression of the reporter gene according to the diagnostic assay of any one of embodiments 1 to 14, wherein activation of the reporter gene is indicative for efficacy of the antitumor CAR-T cell treatment when applied to the subject.


SEQUENCE LISTING (74 sequences)

SEQ ID NO: 1 - protein, 5 aa, Artificial sequence
  Anti-CD20 CDR H1 Kabat
YSWIN

SEQ ID NO: 2 - protein, 17 aa, Artificial sequence
  Anti-CD20 CDR H2 Kabat
RIFPGDGDTDYNGKFKG

SEQ ID NO: 3 - protein, 10 aa, Artificial sequence
  Anti-CD20 CDR H3 Kabat
NVFDGYWLVY

SEQ ID NO: 4 - protein, 16 aa, Artificial sequence
  Anti-CD20 CDR L1 Kabat
RSSKSLLHSNGITYLY

SEQ ID NO: 5 - protein, 7 aa, Artificial sequence
  Anti-CD20 CDR L2 Kabat
QMSNLVS

SEQ ID NO: 6 - protein, 9 aa, Artificial sequence
  Anti-CD20 CDR L3 Kabat
AQNLELPYT

SEQ ID NO: 7 - protein, 407 aa, Artificial sequence
  Anti-CD20-Fab-heavy chain-CD28ATD-CD28CSD- CD3zSSD fusion
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 222 aa, Artificial sequence
  Anti-CD20-Fab-heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 219 aa, Artificial sequence
  Anti-CD20-Fab-light chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 112 aa, Artificial sequence
  Anti-CD20-VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 11 - protein, 107 aa, Artificial sequence [sequence omitted]

SEQ ID NO: 12 - protein, 119 aa, Artificial sequence
  Anti-CD20-VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 13 - protein, 103 aa, Artificial sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 27 aa, Artificial sequence
  CD28ATD
FWVLVVVGGVLACYSLLVTVAFIIFWV

SEQ ID NO: 15 - protein, 41 aa, Artificial sequence
  CD28CSD
RSKRSRLLHSDYMNMTPRRPGPTRKHYQPYAPPRDFAAYRS

SEQ ID NO: 16 - protein, 112 aa, Artificial sequence
  CD3zSSD
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 180 aa, Artificial sequence
  CD28ATD-CD28CSD-CD3zSSD
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 238 aa, Artificial sequence
  eGFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 19 - protein, 20 aa, Artificial sequence
  (G4S)4 linker
GGGGSGGGGSGGGGSGGGGS

SEQ ID NO: 20 - protein, 5 aa, Artificial sequence
  G4S linker
GGGGS

SEQ ID NO: 21 - protein, 19 aa, Artificial sequence
  T2A linker
GEGRGSLLTCGDVEENPGP

SEQ ID NO: 22 - DNA, 2659 bp, Artificial sequence
  Anti-CD20-Fab-CD28ATD-CD28CSD-CD3zSSD
atgggatggagctgtatcatcctcttcttggtagcaacagctaccggtgtgcattccgatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaaccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaatagaattccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaacgccaccatgggatggagctgtatcatcctcttcttggtagcaacagctaccggtgtgcactcccaggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 23 - DNA, 336 bp, Artificial sequence
  Anti-CD20- Fab-VL
gatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaa

SEQ ID NO: 24 - DNA, 324 bp, Artificial sequence
  Fab CL
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 25 - DNA, 357 bp, Artificial sequence
  Anti-CD20- Fab-VH
caggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctccagc

SEQ ID NO: 26 - DNA, 309 bp, Artificial sequence
  Fab CH1
gctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgc

SEQ ID NO: 27 - DNA, 647 bp, Artificial sequence
  IRES EV71 internal ribosomal entry side
cccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaac

SEQ ID NO: 28 - DNA, 15 bp, Artificial sequence
  G4S linker
ggagggggcggatcc

SEQ ID NO: 29 - DNA, 81 bp, Artificial sequence
  CD28ATD
ttttgggtgctggtggtggttggtggagtcctggcttgctatagcttgctagtaacagtggcctttattattttctgggtg

SEQ ID NO: 30 - DNA, 123 bp, Artificial sequence
  CD28CSD
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 31 - DNA, 336 bp, Artificial sequence
  CD3zSSD
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - DNA, 540 bp, Artificial sequence
  CD28ATD-CD28CSD-CD3zSSD
ttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 33 - DNA, 63 bp, Artificial sequence
  T2A linker
tccggagagggcagaggaagtcttctaacatgcggtgacgtggaggagaatcccggccctagg

SEQ ID NO: 34 - DNA, 717 bp, Artificial sequence
  eGFP
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 35 - DNA, 3438 bp, Artificial sequence
  Anti-CD20-Fab-CD28ATD-CD28CSD-CD3zSSD-eGFP
atgggatggagctgtatcatcctcttcttggtagcaacagctaccggtgtgcattccgatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaatagaattccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagtgcctattgagctaactggtagtcctccggcccctgattgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaactgtttacctatttattggttttgtaccattatcactgaagtctgtgatcactctcaaattcattttgaccctcaacacaatcaaacgccaccatgggatggagctgtatcatcctcttcttggtagcaacagctaccggtgtgcactcccaggtgcaattggtgcagtctggcgctgaagttaagaagcctgggagttcagtgaaggtctcctgcaaggcttcgggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcggagggggcggatccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtataacgagctgaacctgggcaggagggaggagtacgacgtgctggacaagaggaggggcagggaccccgagatgggcggcaagcccaggaggaagaacccccaggagggcctgtataacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 36 - protein, 5 aa, Artificial sequence
  Anti-CEA CDR H1 Kabat
EFGMN

SEQ ID NO: 37 - protein, 17 aa, Artificial sequence
  Anti-CEA CDR H2 Kabat
WINTKTGEATYVEEFKG

SEQ ID NO: 38 - protein, 12 aa, Artificial sequence
  Anti-CEA CDR H3 Kabat
WDFAYYVEAMDY

SEQ ID NO: 39 - protein, 11 aa, Artificial sequence
  Anti-CEA CDR L1 Kabat
KASAAVGTYVA

SEQ ID NO: 40 - protein, 7 aa, Artificial sequence
  Anti-CEA CDR L2 Kabat
SASYRKR

SEQ ID NO: 41 - protein, 10 aa, Artificial sequence
  Anti-CEA CDR L3 Kabat
HQYYTYPLFT

SEQ ID NO: 42 - protein, 436 aa, Artificial sequence
  Anti-CEA (98/99)-scFv-CD28ATD-CD28CSD-CD3zSSD fusion
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGGGSDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 251 aa, Artificial sequence
  Anti-CEA (98/99)-scFv
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGGGSDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRT

SEQ ID NO: 44 - protein, 121 aa, Artificial sequence
  Anti-CEA (98/99)-VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 45 - protein, 110 aa, Artificial sequence
  Anti-CEA (98/99)-VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRT

SEQ ID NO: 46 - protein, 10 aa, Artificial sequence
  Anti-WT1 (11D06) CDR H1 Kabat
GGTFSSYAIS

SEQ ID NO: 47 - protein, 17 aa, Artificial sequence
  Anti-WT1 (11D06) CDR H2 Kabat
GIIPIFGTANYAQKFQG

SEQ ID NO: 48 - protein, 11 aa, Artificial sequence
  Anti-WT1 (11D06) CDR H3 Kabat
SIELWWGGFDY

SEQ ID NO: 49 - protein, 11 aa, Artificial sequence
  Anti-WT1 (11D06) CDR L1 Kabat
RASQSISSWLA

SEQ ID NO: 50 - protein, 7 aa, Artificial sequence
  Anti-WT1 (11D06) CDR L2 Kabat
DASSLES

SEQ ID NO: 51 - protein, 8 aa, Artificial sequence
  Anti-WT1 (11D06) CDR L3 Kabat
QQYEDYTT

SEQ ID NO: 52 - protein, 408 aa, Artificial sequence
  Anti-WT1 (11D06)-Fab-heavy chain-CD28ATD- CD28CSD-CD3zSSD fusion
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 257 aa, Artificial sequence
  Anti-WT1 (11D06)-Fab-heavy chain
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 54 - protein, 212 aa, Artificial sequence
  Anti-WT1 (11D06)-light chain
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 106 aa, Artificial sequence
  Anti-WT1 (11D06)-VL
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIK

SEQ ID NO: 56 - protein, 120 aa, Artificial sequence
  Anti-WT1 (11D06)-VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSS

SEQ ID NO: 57 - protein, 10 aa, Artificial sequence
  Anti-WT1 (33H09) CDR H3 Kabat
GSYDLFSLDY

SEQ ID NO: 58 - protein, 8 aa, Artificial sequence
  Anti-WT1 (33H09) CDR L3 Kabat
QQYYDGIT

SEQ ID NO: 59 - protein, 434 aa, Artificial sequence
  Anti-WT1 (33H09)-scFv-CD28ATD-CD28CSD-CD3zSSD fusion
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGSYDLFSLDYWGQGTTVTVSSGGGGSGGGGSGGGGSGGGGSDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 245 aa, Artificial sequence
  Anti-WT1 (33H09)-scFv
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGSYDLFSLDYWGQGTTVTVSSGGGGSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYYDGITFGQGTKVEIK

SEQ ID NO: 61 - protein, 119 aa, Artificial sequence
  Anti-WT1 (33H09)-VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGSYDLFSLDYWGQGTTVTVSS

SEQ ID NO: 62 - protein, 106 aa, Artificial sequence
  Anti-WT1 (33H09)-VL
DIQMTQSPSTLSASVGDRVTITCRASQSISSWLAWYQQKPGKAPKLLIYDASSLESGVPSRFSGSGSGTEFTLTISSLQPDDFATYYCQQYYDGITFGQGTKVEIK

SEQ ID NO: 63 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - DNA, 492 bp, Homo sapiens
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 65 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 66 - DNA, 495 bp, Mus musculus
atgaagtggaaagtgtctgttctcgcctgcatcctccacgtgcggttcccaggagcagaggcacagagctttggtctgctggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 67 - DNA, 660 bp, Homo sapiens
atgctgcgcctgctgctggcgctgaacctgtttccgagcattcaggtgaccggcaacaaaattctggtgaaacagagcccgatgctggtggcgtatgataacgcggtgaacctgagctgcaaatatagctataacctgtttagccgcgaatttcgcgcgagcctgcataaaggcctggatagcgcggtggaagtgtgcgtggtgtatggcaactatagccagcagctgcaggtgtatagcaaaaccggctttaactgcgatggcaaactgggcaacgaaagcgtgaccttttatctgcagaacctgtatgtgaaccagaccgatatttatttttgcaaaattgaagtgatgtatccgccgccgtatctggataacgaaaaaagcaacggcaccattattcatgtgaaaggcaaacatctgtgcccgagcccgctgtttccgggcccgagcaaaccgttttgggtgctggtggtggtgggcggcgtgctggcgtgctatagcctgctggtgaccgtggcgtttattattttttgggtgcgcagcaaacgcagccgcctgctgcatagcgattatatgaacatgaccccgcgccgcccgggcccgacccgcaaacattatcagccgtatgcgccgccgcgcgattttgcggcgtatcgcagc

SEQ ID NO: 68 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 69 - DNA, 654 bp, Mus musculus
atgaccctgcgcctgctgtttctggcgctgaacttttttagcgtgcaggtgaccgaaaacaaaattctggtgaaacagagcccgctgctggtggtggatagcaacgaagtgagcctgagctgccgctatagctataacctgctggcgaaagaatttcgcgcgagcctgtataaaggcgtgaacagcgatgtggaagtgtgcgtgggcaacggcaactttacctatcagccgcagtttcgcagcaacgcggaatttaactgcgatggcgattttgataacgaaaccgtgacctttcgcctgtggaacctgcatgtgaaccataccgatatttatttttgcaaaattgaatttatgtatccgccgccgtatctggataacgaacgcagcaacggcaccattattcatattaaagaaaaacatctgtgccatacccagagcagcccgaaactgttttgggcgctggtggtggtggcgggcgtgctgttttgctatggcctgctggtgaccgtggcgctgtgcgtgatttggaccaacagccgccgcaaccgcctgctgcagagcgattatatgaacatgaccccgcgccgcccgggcctgacccgcaaaccgtatcagccgtatgcgccggcgcgcgattttgcggcgtatcgcccg

SEQ ID NO: 70 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 71 - protein, 4 aa, Artificial sequence
  CD28 YMNM
YMNM

SEQ ID NO: 72 - protein, 4 aa, Artificial sequence
  CD28 PYAP
PYAP

SEQ ID NO: 73 - protein, 21 aa, Artificial sequence
  Signal peptide
ATMGWSCIILFLVATATGVHS

SEQ ID NO: 74 - DNA, 57 bp, Artificial sequence
  Signal peptide DNA sequence
atgggatggagctgtatcatcctcttcttggtagcaacagctaccggtgtgcactcc